(19) United States
(12) Patent Application Publication
(10) Pub. No.: US 2025/0140375 A1
(43) Pub. Date: May 1, 2025

(54) METHODS AND DEVICES FOR IMPROVING SLEEP QUALITY AND/OR SUBSEQUENT BEHAVIOURAL OUTCOMES

(71) Applicant: SOCIETE DES PRODUITS NESTLE S.A., Vevey (CH)

(72) Inventors: FRANCOIS-PIERRE MARTIN, Vuisternens-devant-Romont (CH); VANESSA CAROLINE CAMPOS, Forel (CH); KONSTANTINOS MANTANTZIS, Renens VD (CH)

(21) Appl. No.: 18/693,735

(22) PCT Filed: Sep. 21, 2022
(86) PCT No.: PCT/EP2022/076164

§ 371 Date: Mar. 20, 2024

Related U.S. Application Data
(60) Provisional application No. 63/246,405, filed on Sep. 21, 2021.

(51) Int. Cl. G16H 20/60; A61B 5/00; G16H 10/60

(52) U.S. Cl. CPC G16H 20/60; A61B 5/4809; A61B 5/4815; A61B 5/4848; G16H 10/60

(57) ABSTRACT

An aspect is a method of improving sleep quality and/or subsequent behavioural outcomes. The method includes providing a dietary recommendation by evaluating a parameter of the individual selected from the group consisting of sleep time, gender, age, BMI, PA, glycemic status, food preference, food sensitivity, caloric intake, nutrients, meal glycemic load, time of consumption and combinations thereof, providing a nutritional composition based on the dietary recommendation, and administering the nutritional composition to the individual. The nutritional composition is selected from the group consisting of a first composition comprising alpha-casozepine, a second composition comprising tryptophan, a third composition comprising mulberry leaf extract, and combinations thereof. Another aspect is a non-transitory computer-readable medium storing a program that causes a device to provide a dietary recommendation and order a nutritional composition based on the dietary recommendation. Yet another aspect is a device comprising a non-transitory computer-readable medium disclosed herein.

The present disclosure generally relates to methods and devices for health and well-being. More particularly, the present disclosure relates to methods and devices for improving sleep quality and/or subsequent behavioural outcomes.

### BACKGROUND

Sleep is a crucial biological function and is considered an important driver of health and well-being across the lifespan. Good sleep quality has been associated with benefits for brain functions, mood and mental performance, cardio-metabolic health, as well as immunity, (Alvarez et al., 2004) whilst poor sleep quality can lead to negative consequences for health and well-being (Hublin et al., 2007).

Typical sleep architecture is comprised of two components: non-rapid eye movement (NREM; Slow Wave Sleep—SWS) and rapid eye movement (REM) sleep. Overall, sleep quality is thought to be driven by the total duration of SWS, whereas both SWS and REM have been found to jointly contribute to next-day benefits, such as improved cognition. SWS and REM are associated with distinct physiological states, including different requirements in nocturnal energy metabolism, substrate oxidation and blood glucose management.

Sleep quality is strongly associated with cognitive functioning, mood, and feelings of vitality and energy the next day. From a scientific perspective, sleep has been consistently linked with cognitive and mood benefits in humans (for reviews, see Palmer & Alfano, 2017; Rasch & Born, 2013; Walker, 2009). Among the different sleep stages, it has been suggested that SWS duration is more closely linked to declarative memory, whereas REM sleep underlies the ability to synthesize abstract information such as detecting patterns in newly acquired information (non-declarative; Rasch & Born, 2013; Walker, 2009). More recent views on the role of each sleep stage have suggested that SWS and REM might have complementary roles in the consolidation of newly acquired information (for different theories, see Rasch & Born, 2013).

The majority of evidence on the role of sleep for next-day performance comes from sleep deprivation studies, with evidence suggesting that both sleep disruption and sleep deprivation can negatively impact aspects of cognition, including declarative memory, memory encoding and recall, and cognitive flexibility in using existing information to combine them in novel ways (Walker, 2009). Among the cognitive domains strongly influenced by sleep, levels of daytime vigilance and subjective alertness are highly correlated with sleep duration (Jewett et al., 1999). In fact, vigilance tasks have been consistently used as highly sensitive measures of sleep loss (Basner & Dinges, 2011). Furthermore, sleep difficulties can have significant negative effects on emotion regulation through a range of mechanisms, including reduced ability to downregulate amygdala activation to negative information.

Specifically, studies have found that sleep deprivation can cause an increase of almost 60% in amygdala activity when participants are presented with negative images (review, Palmer & Alfano, 2017).

The association between nocturnal glycemia and next-day benefits is less well-understood. Experimentally induced nocturnal hypoglycemia during deep sleep, achieved through insulin infusion to stabilize blood glucose levels to 2.2 mmol/L (40 mg/dL), has been associated with worse memory the next day (Jauch-Chara et al., 2007). In a similar manner, other studies manipulating blood glucose levels to remain within the range of 2.3-2.7 mmol/L (42-48 mg/dL) during deep sleep have uncovered lower levels of well-being, conceptualized as self-reported vitality and contentment (minor symptom evaluation profile; King et al., 1998). It should be noted that most studies linking nocturnal glycemia with cognitive/mood benefits have been conducted with diabetic patients. Therefore, there is a significant gap in understanding how nocturnal glycemia is associated with next-day benefits in healthy populations.

To date, no clear evidence exists on the role of evening meal composition for next-day benefits, and the mechanisms through which it might affect subjective and objective cognitive performance and mood.

### SUMMARY

There is a lack of causality studies on blood glucose, carbohydrate metabolism and sleep. The mechanisms underlying the link between evening dietary carbohydrates and sleep quality are yet unclear. Few studies have reported associations between glucose tolerance and sleep parameters under controlled dietary or therapeutical glucose management conditions, and most studies have used fasting parameters to explore the relationship between sleep parameters and glycemic traits.

Overall, while the knowledge on direct effect of nutrients on the brain and their mode of action to promote sleep is developed, there are still some scientific gaps on the impact of dietary deficiencies vs. supplementation (e.g., by food, beverage or supplement) for sleep quality. There is no product showing a link between improved glucose control and better sleep. Therefore, the inventors explored the relationship between nocturnal glucose metabolism, sleep quality and next-day benefits, to better define the nocturnal glucose/carbohydrate profile through a clinical study with state-of-the-art sleep and metabolic measures in healthy adults.

Among the factors posited to influence sleep quality is tryptophan availability, an amino acid that can promote relaxation and facilitate sleep initiation. The macronutrient composition of evening meals, and particularly the carbohydrate-to-protein ratio, has been closely linked to tryptophan's capacity to cross the blood-brain barrier and boost melatonin synthesis, which could facilitate sleep onset.

In fact, meals high in carbohydrates promote higher tryptophan-to-large neutral amino acid ratio by stimulating the uptake of competing amino acids into muscle, thereby allowing tryptophan to more readily cross the blood-brain barrier (Gangwisch et al., 2020; Yokogoshi & Wurtman, 1986). This phenomenon could explain the positive influence of high-carbohydrate meal intake 4 hours before sleep on sleep initiation (Afaghi et al., 2007).

However, despite high-carbohydrate meals promoting easier transition to sleep, the compensatory hyperinsulinemia and counterregulatory hormonal responses can cause sleep fragmentation and decrease sleep quality.

Clinical trials aiming to improve sleep in healthy individuals have routinely administered doses of tryptophan ranging between 500 mg and 7.5 g, either throughout the day or before sleep, to promote relaxation, calmness and better sleep primarily in those who experience sleep difficulties (Silber & Schmitt, 2010).

However, no studies has demonstrated that an intake of tryptohan taken with an evening meal with a low glycemic response would promote better sleep quality and/or promote subsequent behavioural outcomes.

As set forth in greater detail later herein, the inventors identified nutritional solutions for consumption in the evening to thereby promote sleep quality, based on novel scientific evidence on evening macronutrient composition and sleep health. In particular, emerging science has shown the importance of dietary protein and carbohydrate profiles for sleep quality, which are mediated through nocturnal carbohydrate metabolism and brain functions involved in sleep-wake cycle.

A low glycemic index (GI) and fiber-rich evening diet, or a reduced glycemic response to evening meals (e.g., a reduction of 30% in glycemic response, with a glycemic load (GL) of the evening meal being decreased e.g. from 55 to 38.5), will promote better sleep quality and next day benefits in general population with sleep complaints. Yet today, the mechanism of actions remains poorly understood. One identified mode of action may relate to how high glycemic response to evening meals may result in perturbation of nocturnal glucose and carbohydrate metabolism, which can decrease sleep quality. Postprandial hyperglycemia from high dietary glycemic load and resultant compensatory hyperinsulinemia can lower plasma glucose to concentrations that compromise brain glucose (3.8 mmol/L; 68 mg/dL), triggering the secretion of autonomic counterregulatory hormones such as adrenaline, cortisol, glucagon, and growth hormone. Symptoms of counter-regulatory hormone responses can include heart palpitations, tremor, cold sweats, anxiety, irritability, and hunger. In addition, hypoglycemic events have been shown to cause arousals and substantially reduce sleep efficiency even in healthy adults (Gais et al., 2003).

Accordingly, in a non-limiting embodiment, the present disclosure provides a method of improving sleep and/or subsequent behavioural outcomes of an individual in need thereof by a device having one or more processors. The method may comprise receiving an input from the individual. The input may comprise at least one of sleep quality, gender, age, body mass index, physical activity level, glycemic status, food preference, or food sensitivity. The method may comprise evaluating, by the device, a need of the individual. The need may be selected from the group consisting of improving sleep quality, improving falling asleep, improving sleep maintenance, maintaining sleep quality, and combinations thereof. The method may further comprise generating, by the device via an application interface and based on the input from the individual, a nutritional rule and the need of the individual, a recommendation for the individual. The recommendation may comprise at least one of (1) a dietary plan comprising at least one of a recipe, a food item, a food product, or a diet tip, (2) a dietary restriction on at least one of food sensitivity, sleep complain, or sleep comorbidity, (3) a diet optimization of at least one of caloric intake, nutrient, meal glycemic load, or time of consumption, or (4) sleep enhancing recommendation. The method may further comprise assessing the individual's sleep quality. In a particular non-limiting embodiment, the input may further comprise sleep quality, and the method may further comprise adopting the recommendation by the individual; and reassessing, by the device, the individual's sleep quality.

In some embodiments, the dietary plan may be adapted to the individual's preference selected from the group consisting of vegan, vegetarian, lactose-free, gluten-free and combination thereof.

In some embodiments, the recommendation may comprise a multi-day evening meals plan.

In another embodiment, the nutritional rule comprises at least one of (i) an amount and/or profile and/or a food source of a nutrient effective for promoting sleep, or (ii) a glycemic load of a dietary intake. The nutrient may be selected from the group consisting of carbohydrate, tryptophan, magnesium, zinc, tryptophan, B-vitamins, melatonin, glycine, protein, PUFAs, omega 3, and combinations thereof.

In another embodiment, the recommendation may comprise a sleep-enhancing evening meal to be consumed with a nutritional composition. In a preferred embodiment, the nutritional composition may be selected from the group consisting of a first composition comprising alpha-casozepine, a second composition comprising tryptophan, a third composition comprising mulberry leaf extract, and combinations thereof.

In some embodiments, the nutritional composition may comprise the first composition and may be administered along with a meal comprising at least one of (i) tryptophan from about 120 mg up to about 5 g, (ii) a sleep initiation beneficial micronutrient selected from the group consisting of magnesium, zinc, B-vitamins and combinations thereof, or (iii) a sleep functional ingredient is selected from the group consisting of melatonin, gamma aminobutyric acid (GABA), herbal extracts and combinations thereof.

In another embodiment, the nutritional composition may comprise at least one of a glucosidase inhibitor or an amylase inhibitor. The glucosidase inhibitor may be selected from the group consisting of 1-deoxynojirimycin (DNJ), mulberry leaf extract, mulberry fruit extract, phloridzin, arginine-proline (AP) dipeptide and combinations thereof, and the amylase inhibitor may be selected from the group consisting of white kidney bean extract, wheat albumin and combinations thereof.

In yet another embodiment, the nutritional composition may comprise at least one of the second composition or the third composition and is administered along with an evening meal. In a preferred embodiment, the evening meal may comprise tryptophan in a range from about 120 mg to about 500 mg.

In some embodiments, the nutritional composition may be administered to the individual at a predetermined time (from about thirty minutes to about one hour) before consumption of a meal.

In another embodiment, the nutritional composition may be administered to the individual concurrently with consumption of a meal.

Another aspect of the present disclosure is directed to a computer implemented method of improving sleep quality and/or subsequent behavioural outcomes. In a non-limiting embodiment, the method may include providing a dietary recommendation by evaluating a parameter of the individual. The parameter of the individual may be selected from the group consisting of falling asleep time, sleep duration, sleep onset latency, wakeafter sleep onset, sleep efficiency, gender, age, BMI (Body Mass Index), PA (Physical activity), glycemic status, food preference, food sensitivity, caloric intake, nutrients, meal glycemic load, time of consumption and combinations thereof. The method may include providing a nutritional composition based on the dietary recommendation. The method may further include administering the nutritional composition to the individual.

In a preferred embodiment, the nutritional composition is selected from the group consisting of a first composition comprising alpha-casozepine, a second composition comprising tryptophan, a third composition comprising mulberry leaf extract, and combinations thereof.

In a particular non-limiting embodiment, the dietary recommendation may comprise a recommendation for a meal comprising a nutrient selected from the group consisting of magnesium, zinc, tryptophan, B-vitamins, melatonin, glycine, protein, healthy fats, omega 3, fiber and combinations thereof.

In some embodiments, the nutritional composition may be administered to the individual at a predetermined time (from about thirty minutes to about one hour) before consumption of a meal.

In another embodiment, the nutritional composition may be administered to the individual concurrently with consumption of a meal.

Another aspect of the present disclosure is directed to a non-transitory computer-readable medium storing a program for improving sleep and/or subsequent behavioural outcomes of an individual in need thereof. In a non-limiting embodiment, the non-transitory computer-readable medium storing a program that causes a device to provide a dietary recommendation by evaluating a parameter of the individual. The parameter of the individual may be selected from the group consisting of sleep time, gender, age, BMI, PA, glycemic status, food preference, food sensitivity, caloric intake, nutrients, meal glycemic load, time of consumption and combinations thereof. The program may cause a device to order a nutritional composition based on the dietary recommendation. Ordering a nutritional composition may comprise identifying a product and/or products to purchase. Ordering a nutritional composition may also comprise providing payment method and/or information to a website and/or an application. Ordering a nutritional composition may also comprise providing delivery address or any other information and/or step to fulfill an order as required by the website and/or application. The nutritional composition may be selected from the group consisting of a first composition comprising alpha-casozepine, a second composition comprising tryptophan, a third composition comprising mulberry leaf extract and combination thereof.

In a particular non-limiting embodiment, the dietary recommendation may comprise a recommendation for a meal comprising a nutrient selected from the group consisting of magnesium, zinc, tryptophan, B-vitamins, melatonin, glycine, protein, healthy fats, omega 3, fiber and combinations thereof.

In some embodiments, the nutritional composition may be administered to the individual at a predetermined time (from about thirty minutes to about one hour) before consumption of a meal.

In another embodiment, the nutritional composition may be administered to the individual concurrently with consumption of a meal.

Another aspect of the present disclosure is directed to a device for improving sleep and/or subsequent behavioural outcomes of an individual in need thereof. In a non-limiting embodiment, the device comprises a memory storing a program for improving sleep and/or subsequent behavioural outcomes and a processor. The program causes the processor to provide a dietary recommendation by evaluating a parameter of the individual. The parameter of the individual may be selected from the group consisting of sleep time, gender, age, BMI, PA, glycemic status, food preference, food sensitivity, caloric intake, nutrients, meal glycemic load, time of consumption and combinations thereof. The program may cause a device to order a nutritional composition based on the dietary recommendation. Ordering a nutritional composition may comprise identifying a product and/or products to purchase. Ordering a nutritional composition may also comprise providing payment method and/or information to a website and/or an application. Ordering a nutritional composition may also comprise providing delivery address or any other information and/or step to fulfill an order as required by the website and/or application. The nutritional composition may be selected from the group consisting of a first composition comprising alpha-casozepine, a second composition comprising tryptophan, a third composition comprising mulberry leaf extract and combination thereof.

In a particular non-limiting embodiment, the dietary recommendation comprises a recommendation for a meal comprising a nutrient selected from the group consisting of magnesium, zinc, tryptophan, B-vitamins, melatonin, glycine, protein, healthy fats, omega 3, fiber and combinations thereof.

In some embodiments, the nutritional composition is administered to the individual at a predetermined time (from about thirty minutes to about one hour) before consumption of a meal.

In another embodiment, the nutritional composition is administered to the individual concurrently with consumption of a meal.

Another aspect of the present disclosure provides a method of improving sleep quality and/or subsequent behavioural outcomes. The method comprises orally administering a composition to an individual at a predetermined time before consumption of a meal and/or concurrently with consumption of a meal. The combination of the composition and the meal has a glycemic load lower than the glycemic load of the meal by itself. For example, the meal the combination of the composition and the meal has a glycemic load that is about 0.0 to about 45.0, for example about 11.0 to about 45.0, preferably about 20 to about 45, and lower than that of the meal by itself.

In another embodiment, the present disclosure provides a method of treating, preventing, and/or reducing at least one of risk, incidence or severity of at least one condition for which improved sleep quality is beneficial. The method comprises orally administering a composition to an individual at a predetermined time before consumption of a meal and/or concurrently with consumption of a meal. The combination of the composition and the meal has a glycemic load lower than the glycemic load of the meal by itself. For example, the combination of the composition and the meal has a glycemic load that is about 0.0 to about 45.0, for example about 11.0 to about 45.0, preferably about 20 to about 45, and lower than that of the meal by itself.

In any embodiment disclosed herein, preferably the meal is an evening meal, for example a balanced evening meal. Preferably, the composition comprises an ingredient that lowers glycemic response in the individual. Optionally the total amount of the ingredient in the composition and any of the ingredient in the meal is effective to promote better sleep quality for the individual.

In some embodiments, the ingredient that lowers glycemic response is one or more of tryptophan (e.g., as a free amino acid and/or in a protein such as whey protein), a glucosidase inhibitor such as 1-deoxynojirimycin (DNJ) (e.g., isolated or in a mulberry leaf or fruit extract) or phloridzin (e.g., isolated or in an apple extract), arginine-proline (AP) dipeptide (e.g., isolated or in a milk protein hydrolysate), a fiber, a resistant starch, a beta-glucan, A-cyclodextrin, glucosidase (e.g., isolated and/or as part of a composition such as mulberry leaf extract), a polyphenol (such as anthocyanins), or an amylase inhibitor (e.g., isolated and/or in a composition such as white kidney bean or wheat albumin).

In some embodiments, the ingredient comprises tryptophan and optionally further comprises a mulberry extract (ME), preferably a mulberry leaf extract (MLE).

In some embodiments, the composition is administered in a unit dosage form comprising about 120 mg to about 250 mg of tryptophan. Optionally the total amount of the tryptophan in the composition and any of the tryptophan in the meal is effective to promote better sleep quality to an individual.

The inventors recognized that whole meal replacements to be consumed everyday may result in low consumer compliance to improve sleep. Instead, a particularly advantageous embodiment disclosed herein provides a composition (e.g., a food, a beverage such as a beverage powder or a liquid beverage, or a supplement) to consume with an evening meal, and the composition reduces the glycemic response to the evening meal to thereby promote sleep quality. The compositions and methods disclosed herein can improve sleep quality by improving nocturnal glycemia during the first hours of sleep (e.g., SWS) which is paramount for promoting the restorative benefits of sleep.

In a particular non-limiting embodiment, the present disclosure provides a product that is a low caloric, low volume (preferably about 100 mL to 250 mL) nutritional solution, and the product combines (i) one or more ingredients lowering glycemic response to evening meals to thereby promote sleep quality, (ii) a protein source rich in bioavailable tryptophan to thereby promote sleep quality, and (iii) one or more supporting ingredients contributing to sleep initiation.

In some embodiments, the product is provided as a dairy powder stick to be reconstituted in a water/dairy diluent, or provided as a plant-based beverage; and the product is orally consumed together with a standardized evening meal. The product and the meal can be consumed between about three (3) hours before bedtime and at least about four (4) hours before bedtime.

Additional features and advantages are described in, and will be apparent from, the following Detailed Description and the Figures.

### BRIEF DESCRIPTION OF DRAWINGS

FIG. 1 is a table showing protein analysis of the experimental products in the experimental example disclosed herein. WPI: whey protein isolate; WPM: whey protein microgel pre-meal; and CGMP: caseinoglycomacropeptide.

FIG. 2 is a table showing macronutrient composition of the standard meals accompanying whey protein drinks and mulberry leaf extract (kcal percentage) in the experimental example disclosed herein. Study 1: whey protein pre-meal. Study 2: mulberry leaf extract.

FIG. 3 is a table showing mean and SEM values for PPGR parameters for all interventions in the experimental example disclosed herein.

FIGS. 4A-D show postprandial glucose excursion by consumption of whey protein 30 min before the meal in the experimental example disclosed herein. Specifically, FIG. 4A is a graph showing postprandial glucose excursion (in mM) over time (in min) for the 3 groups: control (white dots), WPI (grey triangles) or WPM (black dots). FIG. 4B is a graph showing 2 h-incremental area-under-the-curve (iAUC 2 h) of the three groups. FIG. 4C is a graph showing incremental maximal glucose concentration (iCmax) in mM of the three groups. FIG. 4D is a graph showing Time at which maximum postprandial glucose response is reached (Tmax, min). All data is presented as means and standard error of mean (SEM). Asterisks (*) denote significant differences between control and intervention groups (p<0.05), while $ denotes significant difference (p<0.05) between the WPI and WPM groups.

FIGS. 5A-D show postprandial glucose excursion by consumption of whey protein 10 min before the meal. FIG. 5A is a graph showing postprandial glucose excursion (in mM) over time (in min) for the 3 groups: control (white dots), WPI (grey triangles) or WPM (black dots). FIG. 5B is a graph showing 2 h-incremental area-under-the-curve (iAUC 2 h) of the three groups. FIG. 5C is a graph showing incremental maximal glucose concentration (iCmax) in mM of the three groups. FIG. 5D is a graph showing time at which maximum postprandial glucose response is reached (min). All data is presented as means and standard error of mean (SEM). Asterisks (*) denote significant differences between control and intervention groups (p<0.05), while $ denotes significant difference (p<0.05) between the WPI and WPM groups.

FIGS. 6A-D show postprandial glucose excursion by adding MLE before or mixed within a meal. FIG. 6A is a graph showing postprandial glucose excursion (in mM) over time (in min) for the 3 groups: water 5 min before a standardized balanced meal “Control (white dots)”; MLE diluted in water, consumed 5 min before a standardized balanced meal “MLE Before (grey triangles)”; and MLE consumed with a standardized balanced meal “MLE During (black dots).” FIG. 6B is a graph showing 2 h-incremental area-under-the-curve (iAUC 2 h) of the three groups. FIG. 6C is a graph showing incremental maximal glucose concentration (iCmax) in mM of the three groups. FIG. 6D is a graph showing time at which maximum postprandial glucose response is reached (min). All data is presented as means and standard error of mean (SEM). Asterisks (*) denote significant differences between control and intervention groups (p<0.05), while $ denotes significant difference (p<0.05) between the “Before” and “During” groups.

FIG. 7 is a diagram showing a computer implemented method of providing dietary recommendations to promote sleep quality and/or subsequent behavioural outcomes according to an embodiment of the present disclosure.

FIG. 8 is a diagram showing a computer implemented method of providing dietary recommendations in combination with a nutritional composition to promote sleep quality and/or subsequent behavioural outcomes according to an embodiment of the present disclosure.

FIG. 9 is a diagram showing a computer implemented method of providing dietary recommendations in combination with a nutritional composition and glycemic load range to promote sleep quality and/or subsequent behavioural outcomes according to an embodiment of the present disclosure.

FIG. 10 is a diagram showing an algorithm development for customized static sleep-friendly meal plans according to an embodiment of the present disclosure.

FIG. 11 illustrates a flow diagram of an example method of determining a glycemic load of a dietary intake option to provide dietary recommendations according to an exemplary embodiment of the present disclosure.

FIG. 12 includes tables showing example corrective factors for nutrients applicable in an example method of determining a glycemic load of a dietary intake option according to an exemplary embodiment of the present disclosure.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. A range defined using “between” is inclusive of the upper and lower endpoints of the range.

Furthermore, all numerical ranges herein include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. Ranges defined using “between” include the referenced endpoints.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.”Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to rodents; aquatic mammals; domestic animals such as dogs and cats (“companion animals”); farm animals such as sheep, pigs, cows and horses; and humans. Where “animal,” “mammal” or a plural thereof is used, these terms also apply to any animal that is capable of the effect exhibited or intended to be exhibited by the context of the passage, e.g., an animal benefitting from improved sleep quality. While the term “individual” or “subject” is often used herein to refer to a human, the present disclosure is not so limited. Accordingly, the term “individual” or “subject” refers to any animal, mammal or human that can benefit from the methods and compositions disclosed herein.

The relative terms “improve,” “promote,” “enhance” and the like refer to the effects of the method disclosed herein on sleep quality, particularly the administration of a composition comprising an ingredient that lowers glycemic response in the individual (e.g., about 120 mg to about 250 mg of tryptophan), at a predetermined time before consumption of an evening meal and/or concurrently with consumption of an evening meal, relative to consumption of an identically formulated meal but without the ingredient that lowers glycemic response provided by the composition. In some embodiments, sleep quality can be quantified by one or both of (a) a total duration of slow wave sleep (SWS) and/or (b) a total duration of rapid eye movement (REM). For example, an improved sleep quality can be established by one or both of a longer total duration of SWS and/or a total duration of REM. In some embodiments, improvement in sleep quality is improvement of one or more of i) sleep efficiency (e.g. measured by actigraphy data); ii) change in sleep latency (e.g actigraphy data); iii) change in wake after sleep onset (e.g. by actigraphy); iv) change in total sleep duration (mins, actigraphy); v) time in bed; vi) minutes spent in bed after waking up. In other embodiments, sleep quality may be assessed by self reporting (e.g. Karolinska Sleepiness Scale (KSS) or Epworth Sleepiness Scale (ESS).

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder. As used herein, an “effective amount” is an amount that treats or prevents a deficiency, treats or prevents a disease or medical condition in an individual, or, more generally, reduces symptoms, manages progression of the disease, or provides a nutritional, physiological, or medical benefit to the individual.

As used herein, “administering” includes another person providing a referenced composition to an individual so that the individual can consume the composition and also includes merely the act of the individual themselves consuming a referenced composition.

The terms “food,” “food product” and “food composition” mean a composition that is intended for ingestion by an individual, such as a human, and that provides at least one nutrient to the individual. “Food” and its related terms include any food, feed, snack, food supplement, treat, meal substitute, or meal replacement, whether intended for a human or an animal. Animal food includes food or feed intended for any domesticated or wild species. In preferred embodiments, a food for an animal represents a pelleted, extruded, or dry food, for example, extruded pet foods such as foods for dogs and cats.

Within the context of the present disclosure, the term “beverage,” “beverage product” and “beverage composition” mean a potable liquid product or composition for ingestion by an individual such as a human and provides water and may also include one or more nutrients and other ingredients safe for human consumption to the individual.

The terms “serving” or “unit dosage form,” as used herein, are interchangeable and refer to physically discrete units suitable as unitary dosages for human and animal subjects, each unit containing a predetermined quantity of the composition comprising an ingredient that lowers glycemic response as disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. The term “additional” ingredient for the compositions disclosed herein does not necessarily imply that the meal consumed with the composition includes a portion of the ingredient that lowers glycemic response; instead, some embodiments of the meal consumed with the composition include a portion of the ingredient that lowers glycemic response, and some embodiments of the meal consumed with the composition lack the ingredient that lowers glycemic response in some embodiments.

### EMBODIMENTS

An aspect of the present disclosure is a method of improving sleep quality and/or subsequent behavioural outcomes. The method comprises orally administering a composition to an individual at a predetermined time before consumption of a meal (e.g., about thirty minutes before the meal to about one hour before the meal) and/or concurrently to consumption of a meal by the individual. The combination of the composition and the meal has a glycemic load lower than the glycemic load of the meal by itself. For example, the combination of the composition and the meal has a lower glycemic load that is about 11 to about 45, preferably about 20 to about 45.

Subsequent behavioural outcomes enhanced by improved sleep quality include one or more of (a) less frequent and/or less severe sleepiness, stress, anxiety, fatigue or depression and/or (b) more and/or better sleep initiation, relaxation, calmness, alertness, cognition, memory, attention, fat utilization, weight management, immunity, or next-day mood.

In another embodiment, the present disclosure provides a method of treating, preventing, and/or reducing at least one of risk, incidence or severity of at least one condition for which improved sleep quality is beneficial. The method comprises orally administering a composition to an individual at a predetermined time before consumption of a meal (e.g., about thirty minutes before the meal to about one hour before the meal) and/or concurrently to consumption of a meal by the individual. The combination of the composition and the meal has a glycemic load lower than the glycemic load of the meal by itself. For example, the combination of the composition and the meal has a lower glycemic load that is about 0.0 to about 45.0.

In any embodiment disclosed herein, preferably the meal is an evening meal, for example a balanced evening meal.

The meal has a glycemic load of about 26.0 to about 58.5, for example at least about 27.0, at least about 28.0, at least about 29.0, at least about 30.0, at least about 31.0, at least about 32.0, at least about 33.0, at least about 34.0, at least about 35.0, at least about 36.0, at least about 37.0, at least about 38.0, at least about 39.0, or at least about 40.0. In some embodiments, the glycemic load of the meal is no greater than about 58.0, no greater than about 57.0, no greater than about 56.0, no greater than about 55.0, no greater than about 54.0, no greater than about 53.0, no greater than about 52.0, no greater than about 51.0, no greater than about 50.0, no greater than about 49.0, no greater than about 48.0, no greater than about 47.0, no greater than about 46.0, or no greater than about 45.0.

The combination of the composition and the meal has a glycemic load lower than the glycemic index of the meal by itself and in the range of about 11.0 to about 45.0, for example at least about 21.0, at least about 22.0, at least about 23.0, at least about 24.0, at least about 25.0, at least about 26.0, at least about 27.0, at least about 28.0, at least about 29.0, or at least about 30.0. In some embodiments, the glycemic load of the combination of the composition and the meal is no greater than about 44.0, no greater than about 43.0, no greater than about 42.0, no greater than about 41.0, no greater than about 40.0, no greater than about 39.0, no greater than about 38.0, no greater than about 37.0, no greater than about 36.0, or no greater than about 35.0.

As another example, the glycemic load of the meal can be reduced at least about 10%, preferably at least about 20%, preferably at least about 30.0%, most preferably at least about 40.0% in the combination of the meal and the composition.

Preferably, the composition comprises an ingredient that lowers glycemic response in the individual. Optionally the total amount of the ingredient in the composition and any of the ingredient in the meal is effective to promote better sleep quality for the individual.

In some embodiments, the ingredient that lowers glycemic response is one or more of tryptophan (e.g., as a free amino acid and/or in a protein such as whey protein), a glucosidase inhibitor such as 1-deoxynojirimycin (DNJ) (e.g., isolated or in a mulberry leaf or fruit extract) or phloridzin (e.g., isolated or in an apple extract), arginine-proline (AP) dipeptide (e.g., isolated or in a milk protein hydrolysate), a fiber, a resistant starch, a beta-glucan, A-cyclodextrin, glucosidase (e.g., isolated and/or as part of a composition such as mulberry leaf extract), a polyphenol (such as anthocyanins), or an amylase inhibitor (e.g., isolated and/or in a composition such as white kidney bean or wheat albumin).

In some embodiments, the composition is administered once a day (e.g., with the evening meal, preferably not at other meals and/or not at other times of the day) for a total duration of at least two weeks.

In some embodiments, the composition is a beverage administered to a human adult with sleep complaints. In some embodiments, the composition is a cereal snack, a beverage (e.g., RTD beverage) containing cereal, a soup, a porridge, a broth, or flan and is administered to a human adult. In some embodiments, the composition is administered to a human toddler.

In some embodiments, the composition is administered in a unit dosage form comprising about 120 mg to about 5 g of tryptophan, preferably about 120 mg to about 1 g of the tryptophan, more preferably about 120 mg to about 250 mg of the tryptophan, most preferably about 120 mg to about 210 mg of the tryptophan.

In such embodiments, the composition preferably comprises a natural source of tryptophan, for example a natural source of tryptophan that has a high tryptophan/large neutral amino acid ratio (TRP/LNAA ratio). In some embodiments, at least a portion of the tryptophan in the composition is provided by one or both of (i) protein in the composition (e.g., animal protein, such as dairy protein, and/or plant protein) and/or (ii) free form tryptophan in the composition.

For example, some embodiments of the composition are administered before the evening meal (e.g., about thirty minutes before the evening meal to about one hour before the evening meal). In such embodiments, the composition can be administered in a unit dosage form comprising whey protein microgels, such as about 9.0 g to about 20.0 g of whey protein microgels, preferably about 9.0 g to about 15.0 g of whey protein microgels, more preferably about 9.0 g to about 11.0 g of whey protein microgels, most preferably about 10.0 g of whey protein microgels. These amounts of whey protein microgels can comprise about 200 mg tryptophan to about 220 mg tryptophan, for example about 210 mg.

As another example, some embodiments of the composition are administered during the evening meal. In such embodiments, the composition can be administered in a unit dosage form comprising whey protein, such as about 5.0 g to about 20.0 g of whey protein, preferably about 5.0 g to about 15.0 g of whey protein, more preferably about 5.0 g to about 10.0 g of whey protein, even more preferably about 5.0 g to about 5.5 g of whey protein, most preferably about 5.1 g of whey protein.

In other particular embodiments of the composition administered during the evening meal, the composition can be administered in a unit dosage form comprising a mixture of whey protein and casein, such as a mixture of about 8:2 whey:casein, and preferably about 5.0 g to about 20.0 g of a mixture of whey protein and casein, more preferably about 5.0 g to about 15.0 g of a mixture of whey protein and casein, even more preferably about 5.0 g to about 10.0 g of a mixture of whey protein and casein, yet more preferably about 5.5 g to about 6.0 g of a mixture of whey protein and casein, most preferably about 5.6 g of a mixture of whey protein and casein.

In yet other particular embodiments of the composition administered during the evening meal, the composition can be administered in a unit dosage form comprising soy protein, such as about 5.0 g to about 20.0 g of soy protein, preferably about 5.0 g to about 15.0 g of soy protein, even more preferably about 5.0 g to about 10.0 g of soy protein, yet more preferably about 5.5 g to about 10.0 g of soy protein, such as about 5.6 g of soy protein in the composition (e.g., administered during an evening meal comprising 50.0 mg tryptophan) or about 9.6 g of soy protein (e.g., administered during an evening meal lacking endogenous tryptophan).

Additionally or alternatively, at least a portion of the protein can be whey protein isolate.

As used herein, “meal” refers to one or more food products consumed at substantially the same time as each other; preferably such that one or more proteins, one or more carbohydrates, one or more fats and at least one micronutrient are provided by consuming the meal; more preferably such that one or more proteins, one or more carbohydrates, one or more fats, one or more vitamins and one or more minerals are provided by consuming the meal. Preferably the meal comprises a plurality of food products. As used herein, “balanced meal” refers to meal which provides all of protein, carbohydrate, fat, vitamins and minerals, in quantities and proportions suitable to maintain health or growth of an individual. The quantities and proportions of protein, carbohydrate, fat, vitamins and minerals suitable to maintain health or growth may be determined in line with the current food and nutrition regulations, and any specific requirements of the individual, for example based on age, physical activity, and/or gender.

For example, the Food and Nutrition Board of the Institutes of Medicine (IOM) current energy, macronutrient, and fluid recommendations, recommend an acceptable macronutrient distribution range for carbohydrate (45%-65% of energy), protein (10%-35% of energy), and fat (20%-35% of energy) for active individuals. In an embodiment, the balanced meal provides 45-65% of total calories from carbohydrate, 20-35% of total calories from fat and of total calories 10-35% from protein. In an embodiment, the meal provides 200 kcal to 1,000 kcal to the individual, preferably 250 kcal to 900 kcal, more preferably 300 kcal to 850 kcal, and most preferably 350 kcal to 800 kcal.

In some embodiments, “evening meal” means a meal consumed about 1.0 hours to about 6.0 hours before the onset of sleep, preferably a meal about 2.0 hours to about 5.0 hours before the onset of sleep, more preferably a meal about 2.5 hours to about 4.5 hours before the onset of sleep, most preferably about 3.0 hours to about 4.0 hours before the onset of sleep.

In some embodiments, “evening meal” means a meal consumed at about 4:30 pm to about 11:30 pm in the geographic region where the individual is located, preferably a meal consumed at about 5:00 pm to about 11:00 pm in the geographic region where the individual is located, more preferably a meal consumed at about 5:30 pm to about 10:30 pm in the geographic region where the individual is located, most preferably a meal consumed at about 6:00 pm to about 10:00 pm in the geographic region where the individual is located.

As used herein, the composition comprising the ingredient that lowers glycemic response is “concurrently” administered with the evening meal if the composition comprising the ingredient that lowers glycemic response is administered between consumption of an initial portion of an initial food product in the meal and consumption of a final portion of a final food product. The composition comprising the ingredient that lowers glycemic response is also “concurrently” administered with the evening meal if the composition comprising the ingredient that lowers glycemic response is administered no more than about five minutes before consumption of an initial portion of an initial food product in the meal, preferably no more than about one minute before consumption of an initial portion of an initial food product in the meal, and no more than about five minutes after consumption of a final portion of a final food product, preferably no more than about one minute after consumption of a final portion of a final food product.

In some embodiments, the composition comprises tryptophan and preferably further comprises a mulberry extract (ME), preferably a mulberry leaf extract (MLE). In such embodiments, the total amount of the tryptophan in the composition and any tryptophan in the balanced meal is effective to promote better sleep quality to an individual.

Preferably the composition is orally administered to the individual in a form selected from the group consisting of a dairy beverage and a non-dairy beverage, and the unit dosage form is a predetermined amount of the beverage (e.g., a predetermined amount of the beverage that comprises about 120 mg to about 250 mg of tryptophan).

In some embodiments, the composition can be a ready to drink (RTD) beverage in a container, and the unit dosage form is a predetermined amount of the RTD beverage sealed in the container, which is opened for the oral administration. For example, the predetermined amount of the RTD beverage can comprise about 120 mg to about 250 mg of tryptophan. An RTD beverage is a liquid that can be orally consumed without addition of any further ingredients. The RTD beverage can be low caloric and/or low volume (e.g., about 100 mL to about 250 mL).

In other embodiments, the method comprises forming the composition by reconstituting a unit dosage form of a powder comprising the ingredient that lowers glycemic response, in water or milk to thereby form the composition subsequently orally administered to the individual (e.g., within about ten minutes after reconstitution, within about five minutes after reconstitution, or within about one minute after reconstitution). The unit dosage form of the powder can be sealed in a sachet or other package, which can be opened for the reconstitution and subsequent oral administration. For example, the predetermined amount of the powder can comprise about 120 mg to about 250 mg of tryptophan. The beverage reconstituted from the powder can be low caloric and/or low volume (e.g., about 100 mL to about 250 mL).

The optional mulberry extract can be of any Morus origin, including, but not limited to, White Mulberry (Morus alba L.), Black Mulberry (Morus nigra L.), American Mulberry (Morus celtidifolia Kunth), Red Mulberry (Morus rubra L.), hybrid forms between Morus alba and Morus rubra, Korean Mulberry (Morus australis), Himalayan Mulberry (Morus laevigata), and combinations thereof.

The mulberry extract can be derived from different parts of mulberry tree, including barks (trunk, twig or root), roots, buds, twigs, young shoots, leaves, fruits or a combination thereof. The mulberry extract can be in the form of e.g. dried powders such as dried powders milled from different parts of the tree. The starting plant material of mulberry extracts can be fresh, frozen or dried mulberry materials. The extract may be used as a liquid or dried concentrated solid. Typically, such an extract includes from at least about 1% w/v 1-DNJ and can be administered in the unit dosage form in an amount of about 7.5 mg of 1-DNJ to about 12.5 mg of 1-DNJ.

For example, a particular non-limiting unit dosage form of the composition can comprise about 750 mg of an extract comprising about 1.0% w/v 1-DNJ or about 250 mg of an extract comprising about 5.0% w/v 1-DNJ.

In a preferred embodiment, the mulberry extract (ME) is a mulberry leaf extract (MLE). The unit dosage form of the composition can comprise a dose of about 400 mg to about 800 mg of mulberry leaf extract (MLE).

Mulberry extracts can be prepared by procedures well known in the art. References in this aspect can be made to Chao Liu et al., Comparative analysis of 1-deoxynojirimycin contribution degree to α-glucosidase inhibitory activity and physiological distribution in Morus alba L, Industrial Crops and Products, 70 (2015) p 309-315; Wenyu Yang et al., Studies on the methods of analyzing and extracting total alkaloids in mulberry, Lishizhen Medicine and Material Medical Research, 2008(5); and CN104666427.

Mulberry leaf extracts are also commercially available, such from Karallief Inc, USA; ET-Chem.com, China; Nanjing NutriHerb BioTech Co., Ltd, China; or from Phynova Group Ltd.

In some embodiments, the unit dosage form of the composition comprising the ingredient that lowers glycemic response can further comprise one or more of melatonin, for example as pistachio powder (e.g., about 0.1 to about 0.3 mg melatonin), Vitamins B3 and B6 (e.g., from about 15% NRV to about 2 mg), magnesium (e.g., about 40 mg magnesium), and/or zinc (e.g., from about 15% NRV to about 15 mg). In some embodiments, the composition can further comprise one or more of gamma aminobutyric acid (GABA), alpha-casozepine, or theanine.

The unit dosage form of the composition comprising the ingredient that lowers glycemic response can additionally contain excipients, emulsifiers, stabilizers and mixtures thereof. The composition may include any nutritional or non-nutritional ingredient that adds bulk, and in most instances will be substantially inert, and does not significantly negate the blood glucose benefits of the composition. The filler material most typically includes a fiber and/or carbohydrate having a low glycemic index.

Carbohydrate sources suitable for inclusion in the compositions disclosed herein include those having a low glycemic index, such as fructose and low DE maltodextrins, because such ingredients do not introduce a high glycemic load into the composition. Other suitable components of the composition include any dietary fiber suitable for human or animal use, including soluble and insoluble fiber, especially soluble fibres. Beneficial effects of soluble fibres on glucose response have been widely reported. Non-limiting examples of suitable soluble fibres include FOS, GOS, inulin, resistant maltodextrins, partially hydrolysed guar gum, polydextrose and combinations thereof.

A non-limiting example of a commercially available fiber for the composition include Sunfiber® (Taiyo International, Inc.,), which is a water-soluble dietary fiber produced by the enzymatic hydrolysis of Guar beans; Fibersol 2™ (Archer Daniels Midland Company), which is a digestion resistant maltodextrin; and polydextrose.

In an embodiment, the composition can comprise tryptophan, a mulberry extract and a soluble fibre. In a preferred embodiment, the composition comprises a soluble fibre selected from polydextrose, a resistant maltodextrin (such as the soluble corn fiber Fibersol-2) and combinations thereof.

The composition may also comprise other filler, stabilizers, anti-caking agents, anti-oxidants or combinations thereof.

The composition may further comprise one or more additional components such as minerals; vitamins; salts; or functional additives including, for example, palatants, colorants, emulsifiers, antimicrobial or other preservatives. Non-limiting examples of suitable minerals for the compositions disclosed herein include calcium, phosphorous, potassium, sodium, iron, chloride, boron, copper, zinc, magnesium, manganese, iodine, selenium, chromium, molybdenum, fluoride and any combination thereof. Non-limiting examples of suitable vitamins for the compositions disclosed herein include water-soluble vitamins (such as thiamin (vitamin B1), riboflavin (vitamin B2), niacin (vitamin B3), pantothenic acid (vitamin B5), pyridoxine (vitamin B6), biotin (vitamin B7), myo-inositol (vitamin B8) folic acid (vitamin B9), cobalamin (vitamin B12), and vitamin C) and fat-soluble vitamins (such as vitamin A, vitamin D, vitamin E, and vitamin K) including salts, esters or derivatives thereof.

The individual may be a mammal such as a human, canine, feline, equine, caprine, bovine, ovine, porcine, cervine or a primate. Preferably the individual is a human.

All references herein to treatment include curative, palliative and prophylactic treatment. Treatment may also include arresting progression in the severity of a disease. Both human and veterinary treatments are within the scope of the present disclosure. Preferably the composition is administered in a serving or unit dosage form that comprises a therapeutically effective or prophylactically effective amount of the ingredient that lowers glycemic response.

Another aspect of the present disclosure is directed to a computer implemented method of improving sleep quality and/or subsequent behavioural outcomes. In a non-limiting embodiment, the method includes providing a dietary recommendation by evaluating a parameter of the individual. The parameter of the individual may be selected from the group consisting of sleep time, gender, age, BMI, Physical activity, glycemic status, food preference, food sensitivity, caloric intake, nutrients, meal glycemic load, time of consumption and combinations thereof. The method may also include providing a nutritional composition based on the dietary recommendation. The method may further include administering the nutritional composition to the individual. The nutritional composition may be selected from the group consisting of a first composition comprising alpha-casozepine, a second composition comprising tryptophan, a third composition comprising mulberry leaf extract, and combinations thereof.

As a non-limiting example, the present disclosure discloses a personalized nutritional solution for good sleep. The personalized nutritional solution is provided via a sleep-friendly meal recipe engine for clinically proven effects on sleep quality and next day benefits. The nutritional solution is a scientific evidence based on computerized method to provide dietary recommendations via a sleep-friendly meal recipe engine for individuals such as adults who search to improve their sleep quality.

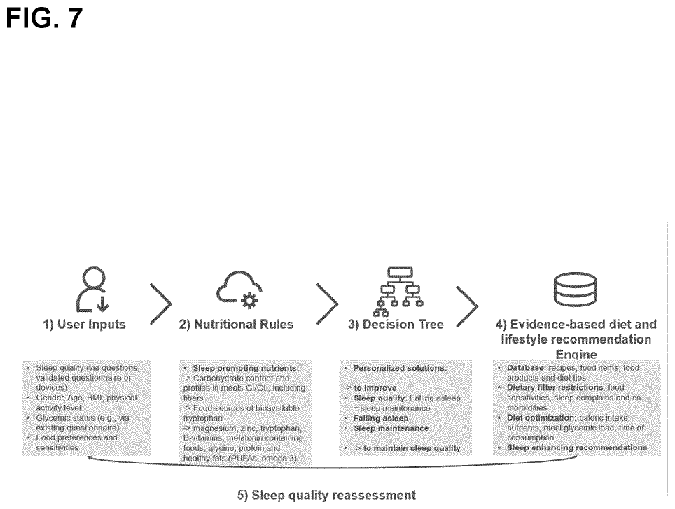

In a particular embodiment, a personalized nutritional solution for good sleep is illustrated in FIG. 7. Firstly, Individual inputs including but not limited to sleep quality assessment, gender, age, BMI, physical activity level, glycemic status, food preferences and sensitivities are provided to a sleep-friendly meal recipe engine. The sleep quality assessment can be obtained by questionnaire, sensor device such as sleep trackers or the like. The sleep quality assessment may also include but not limited to sleep latency, efficiency, Wake After Sleep Onset (WASO), sleep duration overall, deep sleep duration, sleepiness levels and napping during the day. Food preferences may include but not limited to flexitarian, vegetarian, vegan, lactose-free, gluten-free or others. Food sensitivities may include but not limited to lactose, eggs, nuts, shellfish, soy, fish, gluten and others. After the individual inputs are provided to a device and/or a digital platform comprising the sleep-friendly meal recipe engine, the sleep-friendly meal recipe engine is able to generate a dietary recommendation with a nutritional composition based on nutritional rules. The nutritional rules may include recommendations on the sleep promoting nutrients. For example, the sleep promoting nutrients comprise but not limited to carbohydrate content and profiles in meals GI/GL, fibers, food sources of bioavailable tryptophan, magnesium, zinc, tryptophan, B-vitamins, melatonin containing foods, glycine, protein and healthy fats, PUFAs (polyunsaturated fatty acids), omega 3 and combinations thereof.

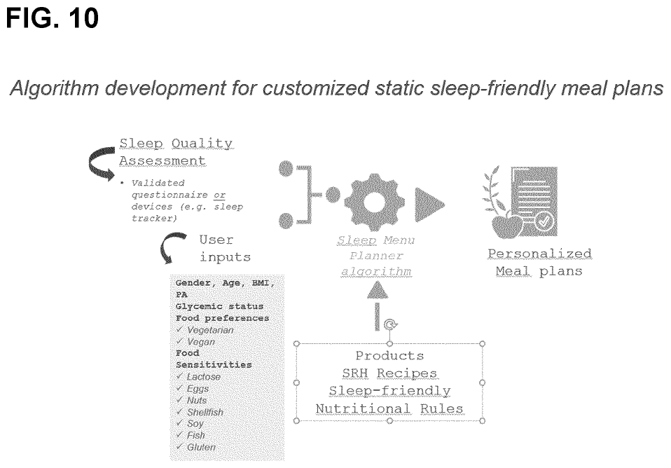

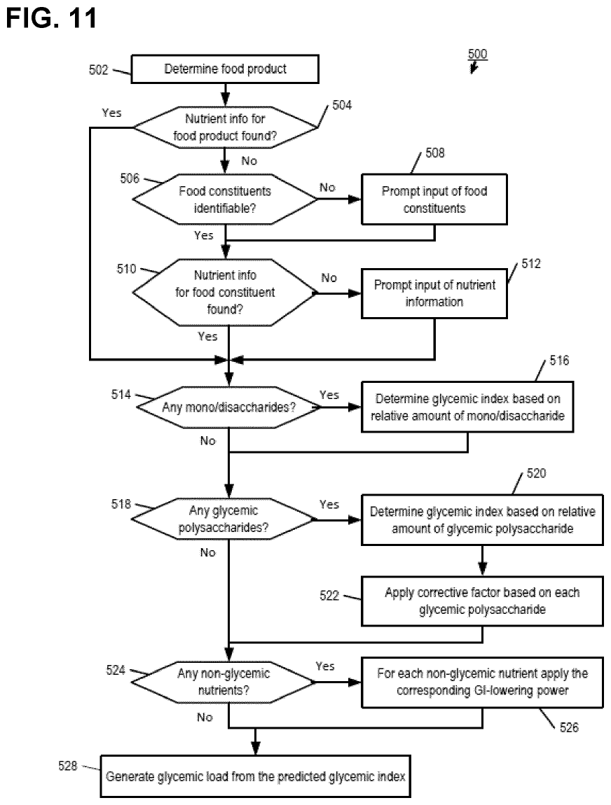

In a preferred embodiment, the nutritional rules may include recommendations on the sleep promoting nutrients in combinations with national dietary guidelines on macronutrient and energy daily needs of male and female subjects, in function of their age and physical activity. The nutritional rules may include calculating a glycemic load of a dietary intake to provide dietary recommendations according to details provided here after in FIGS. 10 and 11. For example, subjects having falling asleep complaints, will be recommended meals and/or snacks with revised GL in combination with ingredient promoting stress and calming before bedtime, such as alpha-casozepine, tryptophan, magnesium, zinc, B-Vitamins, GABA or herbal extracts. For example, subjects having falling sleep maintenance complaints, will be recommended specifically evening meals with specific GL in combination with ingredient improving glucose management (e.g. containing glucosidase inhibitors, such as mulberry leaf or fruit extract (DNJ), phloridzin (apple extract), AP dipeptide (milk protein hydrolysate) or with amylase inhibitors white kidney bean extract, wheat albumin), and/or containing a defined carbohydrate composition (beta-glucans, resistant starch, cyclodextrine, and other Fiber (Fibersol)), in combination with a source rich in tryptophan. The recommendations may be combined with other sleep promoting ingredients. For example, subjects having complaints for falling asleep and sleep maintenance will be recommended a combination of dietary recommendations of the above. For example, subjects having no complaints for falling asleep and sleep maintenance, will be recommended dierary recommenadtions accordingly to national dierary guidelines and with ingredients with healthy GL ranges in combination of sleep promoting ingredients as part of a healthy diet for good sleep.

The sleep-friendly meal recipe engine can also provide personalized, real-time diet and lifestyle recommendations for people seeking to maintain and/or improve their sleep quality. For example, the recommendations can be directed to a target individual that is a child, an adolescent, an adult, or an elderly person. The sleep-friendly meal recipe engine may communicate with a database storing information related to recipes, food items, food products, diet tips and the like. The sleep-friendly meal recipe engine may also be able to generate an evidence-based diet and lifestyle recommendation in view of the information received from the database, dietary filter restrictions such as food sensitivities, sleep complains and comorbidities, and diet optimization. The diet optimization includes but not limited to caloric intake, nutrients, meal glycemic load, and time of consumption. The sleep-friendly meal recipe engine may be able to periodically, at a predetermined time or per individual's request perform sleep quality reassessment to provide an updated diet and lifestyle recommendations based on individuals' needs. The sleep-friendly meal recipe engine may provide a one day meal plan according to individual's need. However, the sleep-friendly meal recipe engine may also provide a multi-day meal plan, preferably a multi-day evening meal plan. The dietary recommendations disclosed herein include a meal solution for maintaining and/or improving sleep quality of an individual in need thereof. The meal solution may be a daily meal or an evening meal, preferably a balanced evening meal comprising sleep promoting nutrients. The dietary recommendation can also be adapted to individual's preferences including but not limited to vegan, vegetarian, lactose-free, gluten-free or the like.

In some embodiments, the sleep-friendly meal recipe engine may be in a form of hardware comprising a CPU (Central Processing Unit) or processor, a peripheral logic part, an interface part, and a storage part such as a RAM (Random Access Memory), a ROM (Read Only Memory), a non-volatile memory and so on. In other embodiments, the sleep-friendly meal recipe engine may be in a form of software and/or application configured to cause a processor to provide customized dietary recommendations for a meal along with a nutritional composition disclosed herein. In yet other particular embodiments, the sleep-friendly meal recipe engine may be a combination of hardware and software or a combination of hardware and application for improving sleep and/or subsequent behavioural outcomes.

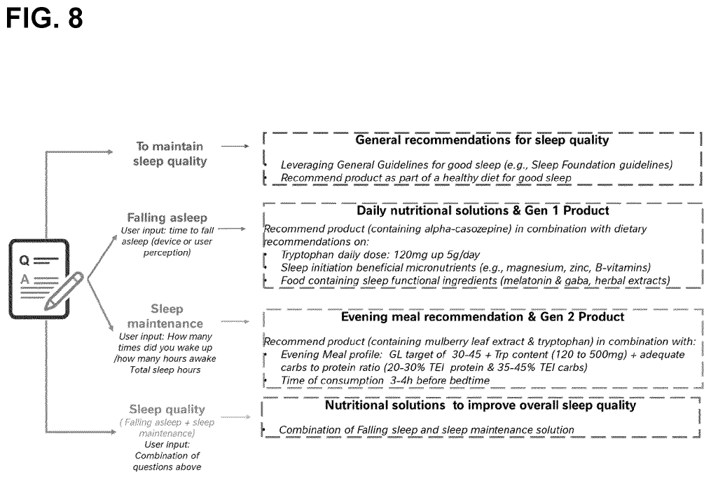

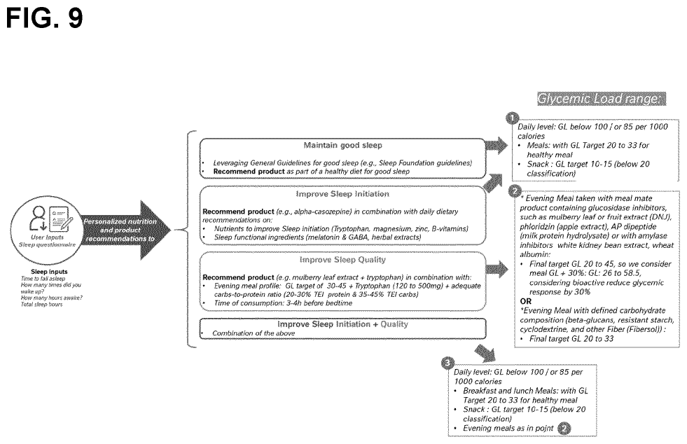

In some embodiments, the sleep-friendly meal recipe engine can provide a dietary recommendation for an individual to maintain sleep quality as illustrated in FIGS. 8 and 9. For example, the sleep-friendly meal recipe engine is configured to provide a general recommendation with a nutritional composition for sleep quality. The general recommendation may be based on general guidelines for good sleep. For example, a balanced diet made up largely of a variety of vegetables and fruits is able to provide the recommended daily intake of vitamins and nutrients, contributing to better sleep while promoting a healthy weight. The nutritional composition may include alpha-casozepine or the like. The nutritional composition can be orally consumed either before or together with a meal based on the general recommendation for good sleep. A daily level of a glycemic load range can be controlled below about 100 per 1000 calories, preferably below about 85 per 1000 calories based on the dietary recommendation along with the nutritional composition.

For example, meals with glycemic load target from about 20 to about 33 for healthy meals, and snacks with glycemic load target from about 10 to about 15 are preferred.

In some embodiments, the sleep-friendly meal recipe engine is able to provide a daily dietary recommendation for an individual to improve sleep quality as illustrated in FIGS. 8 and 9. The improvement of sleep quality can be achieved by decreasing time to fall asleep. The time to fall asleep can either be entered by individuals or be detected by a sensor device such as sleep trackers or the like. For example, the sleep-friendly meal recipe engine is configured to provide a daily dietary recommendation. A nutrional composition comprising a first composition containing alpha-casozepine is administered along with the dail dietary recommendation on tryptophan daily dose, sleep initiation beneficial micronutrients and food containing sleep functional ingredients. For example, the tryptophan daily dose may be in a range of about 120 mg up to 5 g per day.

The sleep initiation beneficial micronutrients may include but not limited to magnesium, zinc or B-vitamins. The sleep functional ingredients may include but not limited to melatonin, gamma aminobutyric acid (GABA) or herbal extracts. A daily level of a glycemic load range can be controlled below about 100 per 1000 calories, preferably below about 85 per 1000 calories based on the daily dietary recommendation along with the nutritional composition.

For example, meals with glycemic load target from about 20 to about 33 are considered as healthy meals, and snacks with glycemic load target from about 10 to about 15 are preferred.

In other particular embodiments, the sleep-friendly meal recipe engine is configured to provide an evening meal recommendation for an individual to improve sleep quality as illustrated in FIGS. 8 and 9. The improvement of sleep quality can also be achieved by enhancing sleep maintenance. The sleep maintenance may be measured in terms of total sleep hours or the like. For example, the sleep-friendly meal recipe engine is configured to provide an evening meal recommendation. The evening meal recommendation may include an evening meal recipe and time of consumption. For example, the evening meal recipe may have a meal profile with glycemic load target from about 30 to about 45, tryptophan content from about 120 to 500 mg, adequate carbohydrate to protein ratio (e.g. 20%-30% TEI protein and 35%-45% TEI carbohydrate). The time of consumption of the meal may be 3 to 4 hours before bedtime. A nutrional composition may be administered along with the evening meal recommendation. The nutrional composition may comprise at least one of mulberry leaf extract or tryptophan according to an embodiment. The nutrional composition may comprise at least one of a glucosidase inhibitor or an amylase inhibitor according to another embodiment. The glucosidase inhibitor is selected from the group consisting of 1-deoxynojirimycin (DNJ), mulberry leaf extract, mulberry fruit extract, phloridzin, arginine-proline (AP) dipeptide and combinations thereof, and the amylase inhibitor is selected from the group consisting of white kidney bean extract, wheat albumin and combinations thereof.

The nutritional composition disclosed herein helps to reduce a glycemic response to evening meals by about 30%, and the glycemic load range of the evening meal is expected from 26 to 58.5. Alternatively, the evening meal may contain defined carbohydrate composition such as beta-glucans, resistant starch, cyclodextrine and other fiber (fibersol). The final glycemic load in combination with the nutritional composition is preferably from 20 to 33.

In yet other particular embodiments, the sleep-friendly meal recipe engine is configured to provide a nutritional solution for an individual to improve overall sleep quality as further illustrated in FIGS. 8 and 9. For example, the sleep-friendly meal recipe engine may provide a nutritional solution that combines both the daily dietary recommendation and the evening meal recommendation as disclosed herein. A daily level of a glycemic load range is expected to be controlled below about 100 per 1000 calories, preferably below about 85 per 1000 calories based on the nutritional solution in combination with the nutritional composition.

For example, breakfast and lunch meals are expected to have a glycemic load target from about 20 to about 33. Snack is expected to have a glycemic load target from about 10 to 15. The evening meal is expected to have a glycemic load target from about 20 to 45, preferably from about 20 to 33.

In some embodiments, the sleep-friendly meal recipe engine is an artificial intelligence engine or algorithm to determine meal/diet plans, sleep-friendly recipes, and/or other food/dietary recommendations by evaluating a parameter of the individual selected from the group consisting of sleep time, gender, age, BMI, Physical activity level, glycemic status, food preference, food sensitivity, caloric intake, nutrients, meal glycemic load, time of consumption and combinations thereof. In some embodiments, the food/dietary recommendation identifies one or more sleep-friendly nutrients for consumption by the individual, such as magnesium, tryptophan, B vitamins, glycine, carbohydrate, protein, or melatonin.

In a particular non-limiting embodiment, the present disclosure provides an artificial intelligence engine/algorithm that is developed for providing customized static sleep-friendly meal plans to individuals or families. As illustrated in FIG. 10, the artificial intelligence engine/algorithm may be developed in view of sleep quality assessment, individual inputs, sleep-friendly nutritional rules, products and/or recipes from a database. The sleep quality assessment can be obtained by questionnaire or sensor device such as sleep tracker or the like. The sleep quality assessment may include but not limited to sleep latency, efficiency, WASO, sleep duration overall, deep sleep duration, sleepiness levels and napping during the day. The individual inputs may include but not limited to gender, age, BMI, Physical activity level, glycemic status, food preferences and food sensitivities. Food preferences may further include flexitarian, vegetarian, vegan, lactose-free, gluten-free or others. Food sensitivities may further include lactose, eggs, nuts, shellfish, soy, fish, gluten and others. After the sleep quality assessment and individual inputs are provided, the artificial intelligence engine or algorithm will be able to generate a personalized meal plan based on sleep-friendly nutritional rules, a nutritional composition and recipes. The nutritional rules may include recommendations on the sleep promoting nutrients. For example, the sleep promoting nutrients comprise but not limited to carbohydrate content and profiles in meals GI/GL, fibers, food sources of bioavailable tryptophan, magnesium, zinc, tryptophan, B-vitamins, melatonin containing foods, glycine, protein and healthy fats, PUFAs, omega 3 and combinations thereof. The nutritional composition is administered along with a meal based on the personalized meal plan. The nutritional composition may comprise a composition containing alpha-casozephine according to an embodiment. In another embodiment, the nutritional composition may include another composition selected from the group consisting of tryptophan, mulberry leaf extract, a glucosidase inhibitor, an amylase inhibitor, and combinations thereof.

Another aspect of the present disclosure is directed to a non-transitory computer-readable medium storing a program for improving sleep and/or subsequent behavioural outcomes of an individual in need thereof. In a non-limiting embodiment, the non-transitory computer-readable medium storing a program that causes a device to provide a dietary recommendation by evaluating a parameter of the individual. The parameter of the individual may be selected from the group consisting of sleep time, gender, age, BMI, Physical activity level, glycemic status, food preference, food sensitivity, caloric intake, nutrients, meal glycemic load, time of consumption and combinations thereof. The program may cause a device to order a nutritional composition based on the dietary recommendation.

Ordering a nutritional composition may comprise identifying a product and/or products to purchase. Ordering a nutritional composition may also comprise providing payment method and/or information to a website and/or an application. Ordering a nutritional composition may also comprise providing delivery address or any other information and/or step to fulfill an order as required by the website and/or application. The nutritional composition may be selected from the group consisting of a first composition comprising alpha-casozepine, a second composition comprising tryptophan, a third composition comprising mulberry leaf extract and combination thereof.

In a particular non-limiting embodiment, the dietary recommendation may comprise a recommendation for a meal comprising a nutrient selected from the group consisting of magnesium, zinc, tryptophan, B-vitamins, melatonin, glycine, protein, healthy fats, omega 3, fiber and combinations thereof.

In some embodiments, the nutritional composition may be administered to the individual at a predetermined time (from about thirty minutes to about one hour) before consumption of a meal.

In another embodiment, the nutritional composition may be administered to the individual concurrently with consumption of a meal.

Yet another aspect of the present disclosure is directed to a device for improving sleep and/or subsequent behavioural outcomes of an individual in need thereof. In a non-limiting embodiment, the device comprises a memory storing a program for improving sleep and/or subsequent behavioural outcomes and a processor. The program causes the processor to provide a dietary recommendation by evaluating a parameter of the individual. The parameter of the individual may be selected from the group consisting of sleep time, gender, age, BMI, Physical activity level, glycemic status, food preference, food sensitivity, caloric intake, nutrients, meal glycemic load, time of consumption and combinations thereof. The program may also cause a device to order a nutritional composition based on the dietary recommendation. Ordering a nutritional composition may comprise identifying a product and/or products to purchase. Ordering a nutritional composition may also comprise providing payment method and/or information to a website and/or an application. Ordering a nutritional composition may also comprise providing delivery address or any other information and/or step to fulfill an order as required by the website and/or application. The nutritional composition may be selected from the group consisting of a first composition comprising alpha-casozepine, a second composition comprising tryptophan, a third composition comprising mulberry leaf extract and combination thereof.

In a particular non-limiting embodiment, the dietary recommendation provided by the device may comprise a recommendation for a meal comprising a nutrient selected from the group consisting of magnesium, zinc, tryptophan, B-vitamins, melatonin, glycine, protein, healthy fats, omega 3, fiber and combinations thereof.

In some embodiments, the nutritional composition may be administered to the individual at a predetermined time (from about thirty minutes to about one hour) before consumption of a meal.

In another embodiment, the nutritional composition may be administered to the individual concurrently with consumption of a meal.

Yet another aspect of the present disclosure is directed to a method of determining and calculating a glycemic load of a dietary intake.

FIG. 11 illustrates a flow diagram of an example method of determining a glycemic load of a dietary intake option to provide dietary recommendations according to an exemplary embodiment of the present disclosure. Method 500 may be performed by one or more processors of an analytics server.

Method 500 may begin with identifying a food product (block 502). For example, the food product may comprise a dietary intake option for which the glycemic load is to be calculated. Also or alternatively, the food product may be identified by a user of the personalized digital platform as being consumed or intended to be consumed by the user. For example, the user may enter an identification of or details regarding the food product via app on user device.

The analytics server may determine whether nutrient information for the food product is available. For example, the analytics server may query its food products database, for the food product to see if there is any stored nutrient information. If no nutrient is stored, the analytics server may determine whether there are any identifiable food constituents. For example, the analytics server may query its food products database to see if there are any food product constituents (e.g., ingredients of the food product) listed under the food product. If there are no identifiable food constituents (e.g., none are stored in the food products database, the analytics server may prompt the user to identify what an identified food product (e.g., peanut butter and jelly sandwich) is made of (block 508). The user may input the food constituents (e.g., two loaves of whole wheat bread, one teaspoon peanut butter, and one teaspoon strawberry jelly) via the app on the user device. If information for food products constituents is found in the food product database, or after the user has provided information on the food product constituents, the analytics server may determine if any nutrient information for the food product constituent can be found in the food product database (block 510). For example, the analytics server may query the food products database for each individual food product constituent (e.g., whole wheat bread, peanut butter, strawberry jelly) to retrieve nutrient information associated with each food product constituent. In some aspect, each food product constituent may be stored under data structures associated with other food products. In such aspects, the analytics server may update the data structure associated with the food product identified in block 502 to enter additional data fields for the food product constituents. Nutrients may include, but are not limited to, fats, proteins, water, vitamins, minerals, complex carbohydrates (e.g., glycemic polysaccharides), simple carbohydrates (monosaccharides, disaccharides, etc., and the like). If nutrient information is not found, the analytics server may prompt the user to input the nutrient information for the various food product constituents (block 512). The user may enter in nutritional information for each food product constituent via the app of the user device.

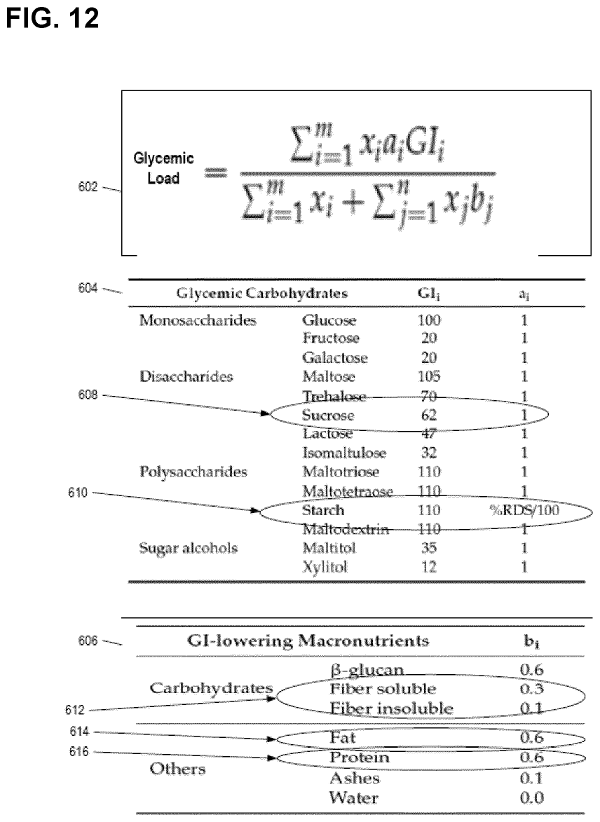

Having retrieved (e.g., from its food products database) or received (e.g., from user device) nutrient information for the food product or the various food product constituents making up the food product, the analytics server may begin analysis of specific nutrients to determine the glycemic load of the food product. For example, the analytics server may determine if the food product has any monosaccharide or disaccharides (block 514). If there are, the analytics server may determine the glycemic index based on the relative amount of the monosaccharide or disaccharide (block 516). As will be discussed herein, FIG. 12 (e.g., via block 602) shows an example of how to determine the glycemic index in more detail.

Afterwards, or if there are no monosaccharides or disaccharides, the analytics server may determine if the food product has any glycemic polysaccharides (block 518). If there are, the analytics server may determine the glycemic index based on the relative amount of the glycemic polysaccharide (block 520). For example, the specific type of glycemic polysaccharide may be identified and the glycemic index may be retrieved from look up tables shown in block 604 of FIG. 12, and which may be stored in the food products database. Furthermore, a corrective factor, aᵢ, based on each glycemic polysaccharide may be applied (block 522). As will be discussed herein, FIG. 12 (e.g., block 604) lists corrective factors, aᵢ, of various known glycemic carbohydrates.

Afterwards, or if there are no glycemic polysaccharides, the analytics server may determine if the food product has any non-glycemic nutrients (block 524). Such non-glycemic nutrients may include, but are not limited to, beta-glucan, fiber, fat, protein, ashes, and water. If there are, the analytics server may apply a GI-lowering power, bᵢ, associated with each non-glycemic nutrient in the determination of the glycemic index (block 526). For example, the specific type of non-glycemic nutrient may be identified from block 606 of FIG. 12, and the glycemic load may be calculated based on methods shown in block 602 of FIG. 12. As will be discussed herein, FIG. 12 (e.g., block 606) lists the GI-lowering powers, bᵢ, of various known non-glycemic nutrients.

Having identified the nutrients and the respective corrective factors, aᵢ, and GI-lowering powers, bᵢ, the analytics server may generate the glycemic load of the food product from the predicted glycemic index (block 528). For example, the analytics server may utilize the equation shown in block 602 of FIG. 12 to calculate the glycemic load.

FIG. 12 includes tables showing example corrective factors for nutrients to be applied in an example method of determining a glycemic load of a dietary intake option according to an exemplary embodiment of the present disclosure. For example, block 602 shows an exemplary method of determining a glycemic load based on the sum of the glycemic index GIᵢ of various nutrients xᵢ. As previously discussed, corrective factors aᵢ may be applied for nutrients such as glycemic carbohydrates. The sum may be divided by the sum of the nutrients plus the sum of any GI-lowering nutrients, xⱼ and their respective GI-lowering powers, bⱼ.

Block 604 is a table listing examples of various glycemic carbohydrates and simple carbohydrates. For example, glycemic polysaccharides include glycemic polysaccharides, and the simple carbohydrates include monosaccharides, disaccharides, and sugar alcohols. The glycemic index, GIᵢ, for these carbohydrates along with any corrective factors, aᵢ, are also listed.

Block 606 is a table listing examples of various GI-lowering nutrients. For example, such GI-lowering nutrients include carbohydrates such as Beta-glucan, fiber, fat, protein, ashes, and water. The GI-lowering powers, bᵢ, for the respective nutrients are also listed.

Furthermore, in at least one embodiment, methods for determining glycemic load and glycemic index may be simplified further. The simplified method may utilize five parameters—sucrose 608, starch 610, fiber 612, fat 614, and protein 616. The simplified method allows for a greater variety of food products to be more efficiently assessed for its glycemic index and/or glycemic load, as nutritional information for the five parameters may be more readily accessible. In some aspects, other nutrients may be linked to or otherwise placed within one or more of the five parameters based on their likeness or similarity in their corrective factor, aᵢ, or GI-lowering power, bᵢ.

### NON-LIMITING EXAMPLES

### Example 1

The following non-limiting example presents experimental data developing and supporting the concepts of embodiments provided by the present disclosure.

### ABSTRACT

### Introduction

Nutritional supplements were reported to decrease glucose response of a meal, including whey protein pre-meals and mulberry leaf extract (MLE). Here, the study evaluated in non-diabetic subjects if the efficacy of these two supplements could be affected by changing timing of consumption or by different whey protein structures.

### Research Design and Methods

Two randomized crossover case-controlled studies were performed. First, fourteen (14) overweight participants consumed 10 g whey protein isolate preparation (WPI) or whey protein microgel solution (WPM) at 30 minutes or 10 minutes before a standard meal. Second, thirty (30) healthy subjects consumed 250 mg of mulberry leaf extract (MLE) before or with a complete balanced meal. Acute postprandial glucose response (PPGR) was monitored with a continuous glucose monitoring (CGM) device.

### Results

In both studies, the different supplements significantly reduced glucose response of the standard meals at all consumption timepoints. While timing of WPI or WPM consumption 30 minutes or 10 minutes before the meal did not affect their efficacy on lowering PPGR, consumption of MLE with the meal resulted in a stronger decrease in PPGR as compared to taking it before the meal (iAUC −16%, p=0.03). For the protein pre-meal, WPM showed a stronger decrease in PPGR compared to WPI, especially when taken 30 minutes before the meal (iAUC −19%, p=0.04).

### CONCLUSIONS

The study confirmed that MLE and whey protein pre-meals are efficient solutions for lowering glucose response of complete meals, and their efficacy can be optimized by choosing best administration timing or protein structure.

### Detailed Research Design and Methods

### Study Design and Subjects

Both studies were monocentric with a crossover, randomized and open design. The number of experimental conditions was six and three for Study 1 and 2 respectively (see below). Subjects were randomly assigned to a sequence of a Williams Latin square that balanced position and carry-over effect to minimize potential bias. Eligible subjects were recruited following completion of a health status questionnaire and a medical screening visit. After enrollment and before starting the experimental visits, a CGM sensor was placed on the non-dominant arm of the subjects. The day before each testing visit, subjects were required to refrain from consuming alcohol and performing strenuous exercise. They were also asked not to take any medication like aspirin or supplement containing Vitamin C that may affect CGM measurements. Since subjects could test all experimental conditions using the same CGM sensor, randomization could be performed without any restriction such as blocking.

### Study 1: Whey Protein Microgel Pre-meal

To evaluate the effect of whey protein pre-meal shots on the glycemic response of a complete meal, fifteen (15) overweight or obese males and females aged 40 years to 65 years were recruited. Key inclusion criteria were a BMI higher than 27 kg/m2, a sedentary lifestyle (no more than 30 min walking per day) and the ability to understand and sign an informed consent form. Key exclusion criteria were any metabolic disease including diabetes or chronic drug intake, known allergy and intolerance to components of the test products, smokers and contraindications to CGM sensor placement (e.g., skin hypersensitivity). Screening of potential participants was performed by the research nurses and validated by the medical responsible. The sample size was deduced from a previous study that included ten (10) healthy young men and showed a significant effect of a 10 g whey protein pre-load on the PPGR of a standard meal. Assuming similar effect size but increased variability due to increased BMI of subjects, the sample size was set to N=15.

### Study 2: Mulberry Leaf Extract

To study the glycemic response following ingestion of MLE, thirty (30) healthy volunteers aged 18 years to 45 years were recruited. Key inclusion criteria were a healthy status, a BMI between 20 and 29.9 kg/m2 and the ability to understand and sign an informed consent form. Key exclusion criteria were food allergy and intolerance to the products, smokers and contraindications to CGM sensor placement (e.g. skin hypersensitivity). Screening of potential participants was performed by the research nurses and validated by the medical responsible. The sample size was deduced from two previous studies that both reported 25% reduction in PPGR of either a rice-based standard meal or a load of 50 g of maltodextrin. Assuming similar effect sizes and variabilities, the calculated effect size was N=30 to reach a power of 80%.

### Experimental Meals

### Study 1: Whey Protein Microgel Pre-Meal

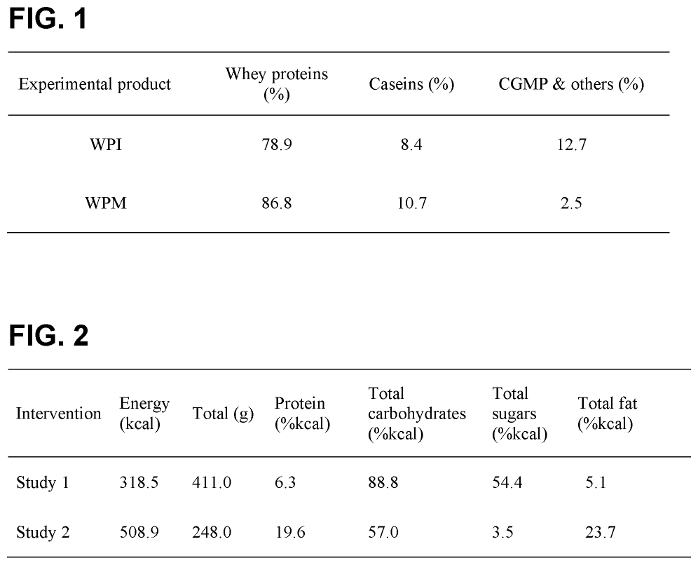

Two drinks containing 10 g of total proteins were compared to a control drink of water. The first drink (WPI) was a whey protein preparation reconstituted in 100 ml of water. The second drink (WPM) was 100 ml of a WPM solution, produced from a native whey protein isolate. For this study, the concentration step was done by conventional evaporation. Whey protein content of WPI and WPM is described in the table in FIG. 1. Each subject consumed a standard breakfast at 10 minutes or 30 minutes after having consumed the experimental products. The breakfast consisted of two slices of white bread (56 g), 25 g of jam, and a glass of orange juice (330 ml). The macronutrient composition of the standard meal is described in the table in FIG. 2.

### Study 2: Mulberry Leaf Extract

250 mg of mulberry (Morus alba) leaf extract (5% Reducose®, Phynova/DSM), containing 12.5 mg of DNJ, was consumed either before (mixed in water) or during (sprinkled over the food) a standard meal composed of 150 g of boiled white jasmine rice, 25 g of white bread, 80 g of curry sauce and 80 g of chicken breast slices. 200 ml of water was consumed before the standard meal. The macronutrient composition of the standard meal is reported in the table in FIG. 2.

### Interventions

In both studies, the participants reached the research center at 8h00 under fasting conditions on the experimental days. Glucose reading with the CGM device was performed just before and after intake of the test products, and interstitial glucose level was continuously and automatically measured every 15 minutes until up to 2 hours after the meal.

### Study 1: Whey Protein Microgel Pre-Meal

In the study evaluating the effects of whey protein, a total of six (6) test visits were necessary for the subjects to complete all experimental interventions:
1. Control 10: 100 ml of water at 10 minutes before the standard meal
2. Control 30: 100 ml of water at 30 minutes before the standard meal
3. WP110: 100 ml of whey protein isolate at 10 minutes before the standard meal
4. WP130: 100 ml of whey protein isolate at 30 minutes before the standard meal
5. WPM10: 100 ml of whey protein microgel at 10 minutes before the standard meal
6. WPM30: 100 ml of whey protein microgel at 30 minutes before the standard meal

### Study 2: Mulberry Leaf Extract

In the study looking at the effects of MLE consumption, the subjects were asked to consume a standardized complete meal within 15 minutes, with one of the 3 arms:
1. Control: 200 ml of water at 5 minutes before the standard meal
2. MLE Before: 250 mg of MLE powder dissolved in 200 ml of water at 5 minutes before the standard meal
3. MLE During: 200 ml of water at 5 minutes before the standard meal, in which 250 mg of MLE powder was mixed

### Measurements

Glucose response was measured with a CGM device, measuring interstitial glucose concentration every 15 minutes. A sensor was installed on the non-dominant arm of each subject at least 24 hours before the first visit; and a reader, as well as instructions for its use, were provided. If a sensor was lost during the study, it was replaced, and the subject could resume the study with the next testing visit, at least 24 hours after sensor insertion. The sensor was removed at the end of the study by a clinical staff member.

### Statistical Analysis

The primary endpoint in these studies was the 2 h-PPGR incremental Area Under the Curve (iAUC) that was calculated using the trapezoid method for each individual PPGR after the standardized meal. Additional endpoints of interest were maximal incremental glucose value (iCmax), the time to reach this value (Tmax), and all cross-sectional timepoints, every 15 minutes after TO. At the beginning of each visit, subjects scanned the sensor with the reader right before and after test product intake and the average was calculated to determine the baseline glycaemia (TO). Descriptive statistics (Mean, SEM) were tabulated and visualized. Means were compared using paired t-tests with significance level set at 5% (two-sided), following established standards. A sensitivity analysis was performed by using a mixed model to impute possible missing data and to consider potential systematic position or carry-over effects. Since none of these effects was close to reach statistical significance, this analysis is not further presented.

### Results

### Baseline Characteristics

### Study 1: Whey Protein Microgel Pre-Meal

Fifteen (15) overweight/obese subjects (6 males, 9 females) were recruited for this study (average age±SEM: 49±8 years, average BMI±SEM: 31.2±2.8 kg/m2) and presented a normal fasting glycaemia (average fasting glucose level±SEM: 5.4±0.6 mM). There was one drop-out due to one participant losing all sensors put on his arm, and 7 visits out of 84 were missed by 6 participants due to loss of the sensor. Due to the drop-out and to the fact that all missing visits could be imputed thanks to the mixed model, the number of subjects to consider in the analyses was N=14.

### Study 2: Mulberry Leaf Extract

The participants (11 males, 19 females) were young (average age±SEM: 31±1.3 years), lean (average BMI±SEM: 22.9±0.4 kg/m2) and normoglycaemic (average fasting glucose level±SEM: 5±0.09 mM). There were no missing visits, but 2 missing data points due to issues with the CGM sensor. None of the subjects reported any side effect of the interventions. The number of subjects to consider in the analyses was N=30.

### Glucose Response

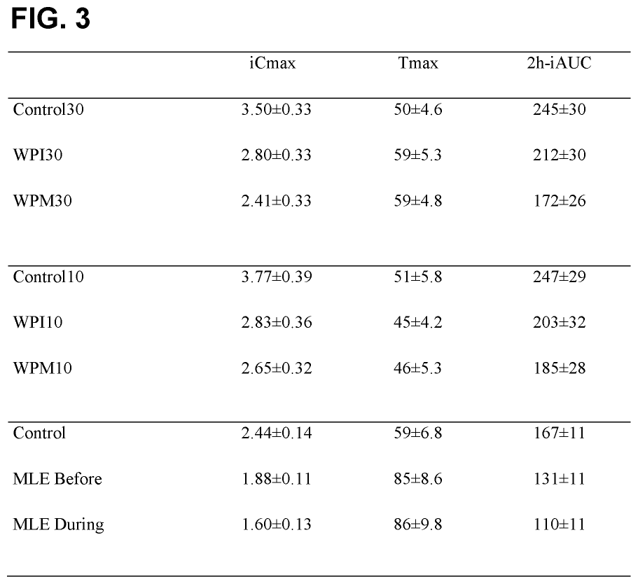

Average PPGR parameters are tabulated for all interventions in the table in FIG. 3.

### Study 1: Whey Protein Microgel Pre-Meal

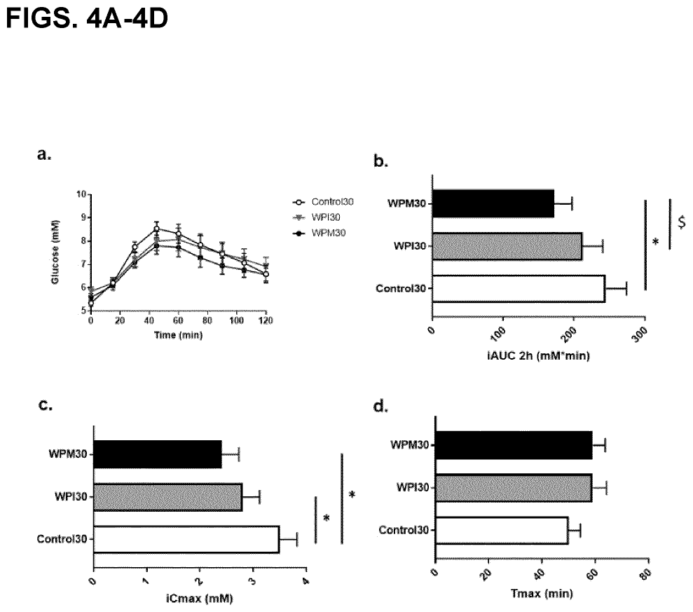

FIG. 4A shows the absolute PPGR values measured during 120 minutes with the CGM device after consumption of WPM and WPI at 30 minutes before the standard breakfast. Compared to the control, WPM30 pre-meal significantly decreased glucose iAUC while only a trend for lowering iAUC was observed with WPI (Mean±SEM effect sizes; WP130: −14±8%, p=0.10; WPM30: −30±7%, p<0.01; FIG. 4B). Both WPM and WPI reduced significantly iCmax of the interstitial glucose curve compared to water as shown in FIG. 4C (WP130: −0.70±0.26 mM, p=0.02; WPM30: −1.09±0.24 mM, p<0.01). Interestingly, glucose iAUC of WPM30 was significantly lower than the one observed with WPI30 (−19±8%, p=0.04).

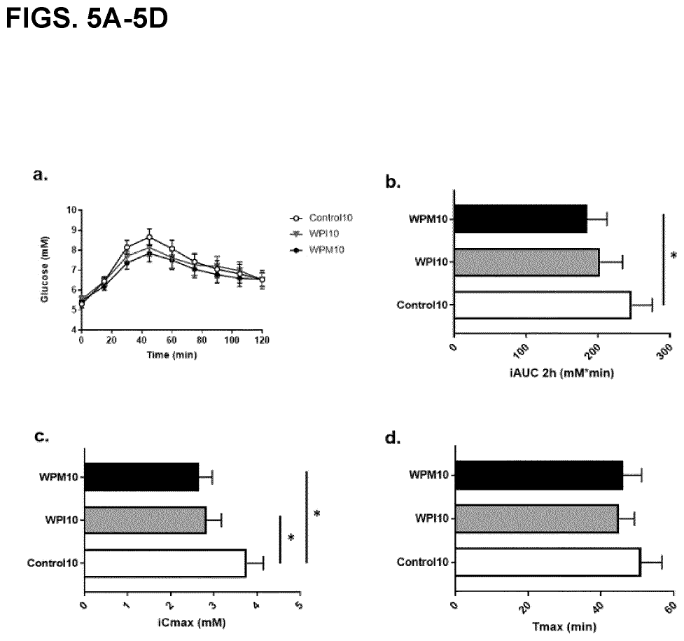

FIG. 5A shows the absolute postprandial glucose values measured during 120 minutes with the CGM device after consumption of WPM and WPI at 10 minutes before the standard breakfast. Glucose iAUC was decreased after WPM consumption and showed only a trend for reduction after WPI (WP110: −18±9%, p=0.08; WPM10: −25±9%, p=0.02; FIG. 5B). Similarly to the observations of the pre-meals taken at 30 minutes before breakfast, when WPM and WPI were consumed at 10 minutes before the standard meal glucose iCmax was significantly lower than after water consumption (WP110: −0.94±0.31 mM, p=0.01; WPM10: −1.13±0.33 mM, p<0.01; FIG. 5C). No significant difference was observed between WPM10 and WPI10 glucose iCmax and iAUC.

Comparing the administration of the whey proteins at 30 minutes versus 10 minutes before the meal, no significant difference was observed in glucose iCmax or iAUC with neither WPM nor WPI. However, interstitial glucose responses after WPM or WPI taken 30 minutes before a meal reached their Tmax later than when taken 10 minutes before (WPI: +14±6 min, p=0.04; WPM: +13±5 min, p=0.03; FIG. 5D versus FIG. 4D).

### Study 2: Mulberry Leaf Extract

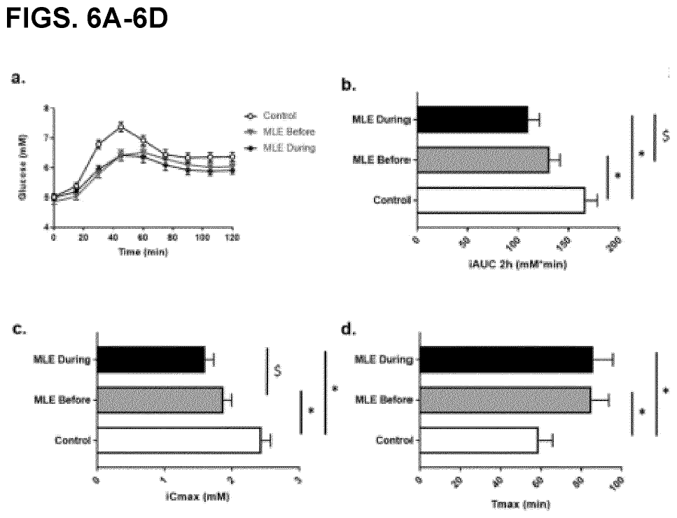

Absolute postprandial interstitial glucose values measured with CGM device in the control and 2 MLE groups are shown in FIG. 6A. Compared to the control, taking MLE before or during the meal reduced PPGR. Plots of the 2 h-iAUC (FIG. 6B) show that taking MLE before the meal significantly attenuated the glucose response by 22±7% (p=0.01), while taking MLE during the meal reduced glucose response by 34±7% (p<0.01). Interestingly, PPGR iAUC of MLE administered with the standardized balanced meal was significantly lower than postprandial glucose iAUC observed for MLE administered before the standardized balanced meal (−16±7%, p=0.03).

Comparing the maximal interstitial PPGR concentrations (FIG. 6C), the iCmax was highest in the Control arm (2.44±0.14 mM). MLE administered both just before and during the standardized meal reduced significantly iCmax of the PPGR curve compared with the control: the MLE Before group (−0.56±0.12 mM, p<0.01) and the MLE During group (−0.84±0.15 mM, p<0.01). The time to reach the maximal glucose concentration (Tmax) was earliest in the Control group (59±7 min) (FIG. 6D) and was also significantly delayed for both the MLE administered just before and during the standardized meal groups compared to the control: the MLE Before group (+26±9 min, p<0.01) and the MLE During group (+28±9 min, p<0.01). Comparing iCmax and Tmax of the group taking the MLE during versus before the standardized meal, there was a significant reduction in iCmax in the MLE During versus MLE Before group (−0.29±0.12 mM, p=0.02) but no difference in Tmax.

### Discussion

This study demonstrated that timing of consumption or protein structure can improve efficacy of MLE or whey protein, respectively, in lowering PPGR. Consumption time (10 minutes or 30 minutes before the meal) did not have any impact on the effects of WPI or WPM pre-meals on the glucose response of the subsequent meal (iAUC and iCmax). These results are consistent with previous results showing that consuming 17.6 g WPI at 15 minutes or 30 minutes before a fat rich meal did not differentially alter PPGR in subjects with metabolic syndrome. The reduction in postprandial interstitial glucose observed in this study was similar to the effect observed on blood glucose response after consumption of 10 g WPI taken 30 minutes before eating a pizza (about −30% in iCmax). This suggests that measurement of interstitial glucose by a CGM device can be used as a good and less invasive alternative to blood sampling. Interestingly, WPM induced a greater reduction in iAUC and Cmax than WPI preload at both consumption times but more importantly when taken 30 minutes before. Because of the delayed protein digestion of WPM compared to WPI, it can be speculated that WPM might induce a stronger GLP-1 stimulation than WPI.

The second study evaluating PPGR effects of MLE confirmed that MLE can decrease PPGR of a complete meal. Compared to an earlier study, in which the same dose of 12.5 mg DNJ (in capsule) in co-ingestion with maltodextrin resulted in 14% reduction of PPGR, the present study observed a similar reduction in PPGR of 16% when the MLE was taken in solution prior to the meal. Similarly, when MLE (8 mg DNJ) was taken with porridge, a 24% reduction of PPGR was reported. Interestingly, the present study demonstrated that timing of administration is an important aspect in obtaining optimal effects of MLE on PPGR. Indeed, MLE induced a stronger reduction of glucose response when mixed with the meal compared to ingestion before the meal. Since DNJ, the active compound in MLE, acts as a competitive α-glucosidase inhibitor, it is reasonable to expect that maximal effect will be observed when the DNJ reaches the small intestine at the same time as the carbohydrates in the food to compete for binding to the α-glucosidase enzymes. In addition to attenuating total PPGR, the present study also observed that consumption of MLE resulted in a later maximal glucose peak (later Tmax). This could mean that the MLE delays absorption of glucose in the gastrointestinal tract and possibly might stimulate GLP-1 secretion. This effect has been observed with another α-glucosidase inhibitor drug, acarbose, where delayed absorption and increased GLP-1 secretion have been demonstrated.

Both WPI pre-meal and MLE have been shown in other studies to reduce PPGR in both diabetics and non-diabetics subjects. Therefore, it is highly likely that the observations in the present study with WPM premeal and MLE will be relevant for diabetic subjects as well.

### Example 2

The following study explored the efficacy of nutritional interventions on sleep quality in healthy adults. This is a double-blind, controlled, randomized, 2-arm, cross-over, group sequential design clinical trial. Subjects will receive the two different nutritional interventions in a randomized order.

### Study Objectives:

Primary objective; To assess the efficacy of the nutritional intervention in improving objective sleep quality among healthy adults with sleep complaints. Primary endpoints: Actigraphy parameters will be used to assess objective sleep quality: i) Change in sleep efficiency (SE), calculated as ‘(total time asleep/time in bed)×100; ii) Change in sleep latency (SOL), measured as the amount of time it takes subjects to fall asleep after going to bed (in minutes)

Secondary objective: To assess the efficacy of the nutritional intervention in improving subjective sleep quality. Endpoints: changes in self-reported sleep quality measured through questionnaires (e.g. Karolinska Sleepiness Scale (KSS) Total sleeping time, wake after onset (WASO).

Trial population: 45 subjects, both male and female between the ages of 25 and 50 with subjective and objective sleep complaints as measured as follows:
Subjective sleep complaints is quantified through sleep quality questionnaires (PSQI>5).
Objective sleep complaints mean sleep efficiency <85% over the 14 days of screening. For this purpose, subjects will be screened during a 2-week screening period using objective sleep monitoring devices (actigraphy).

Treatment administration: The investigational product (IP) was taken in combination with a standardized evening meal (with a glycemic load of 55), orally consumed at least 4 hours before bedtime, and within 30 minutes. The investigational product is consumed once a day for a total duration of two weeks i.e.; 14 days (28 days total for both test and control products).

Test product; beverage to be consumed during the evening meal, containing per serving:
750 mg of mulberry leaf extract containing 1% (m/m) 1-deoxynojirimycin
5.4 g of Whey protein (providing 120 mg of tryptophan)
Fortification in micronutrients: Zinc (1.337 mg), Magnesium (12.39 mg), Vitamins B3 (1.96 mg) and B6 (0.13 mg)

Control product: beverage to be consumed during the evening meal, containing per serving an equivalent protein content low in tryptophan content (4 g of gluten hydrolysate)

The test and control products are provided as powder sachets to be reconstituted in water to a final volume between 200-250 mL.

Treatment and duration: the Investigational product is to be consumed during the meal and will be administered once a day for a total duration of two weeks i.e.; 14 days (28 days total for both test and control products)

The two intervention periods will be separated by a washout period of at least 4 weeks to ensure the subjects return to their baseline sleep status and ensuring no carry over effect, and of at least 6 weeks to ensure female subjects are in the same phase of the menstrual cycle.

During the intervention period, subjects will be provided with customized meals which consist of evening meals, pre-dinner snacks and post dinner beverage. The evening meals are designed based on local dietary guidelines prepared from local commonly consumed foods with a mixture of Asian and western components. The total energy intake (TEI) is based on Estimated Energy Requirements (EER) calculated for adult male and females based on Oxford equation (Henry, 2005). A total of 4 different evening meal menus will be provided to subjects with male and female adapted serving sizes but will otherwise be standardized for macronutrient content. For the evening meals, the profile of the carbohydrates is designed to provide a glycemic load of 55±10%.

Statistical analysis: Continuous variables will be summarized using the appropriate descriptive statistics, including and not limited to: number of observations (n), mean, standard deviation (SD), median, minimum, and maximum.
Primary endpoints: The effect of the intervention on the primary sleep quality parameters (sleep efficiency and sleep latency) will be assessed though a linear mixed-effect model adjusted for the baseline values of the sleep quality parameter
Secondary endpoints: The secondary sleep quality parameters will be analyzed similarly as the primary sleep endpoints.

### Results

### Sleep Efficiency and Sleep Onset Latency

We observed 81% sleep efficiency in total which is explained by a population of test-persons with sleep concerns. After treatment, we observed for sleep efficiency a statistical trend for sleep efficiency improvement (p=0.09) of 1.4% compared to control.

Moreover, Table 1 below reports “sleep onset latency” values at 4-6 days after treatment and 13-14 days of treatment, showing significant positive changes on these days.

TABLE 1
treatment difference of sleep onset latency
in (min) estimated by a mixed model.

| days | Delta | Lo | Up | p-value |
|---|---|---|---|---|
| 1-3 | 0.5 | −8.6 | 9.6 | 0.9159 |
| 4-6 | −9.8 | −18.9 | −0.7 | 0.0342 |
| 13-14 | −11.8 | −20.9 | −2.8 | 0.0112 |

### Secondary Outcomes

The point estimate of total sleeping time is positive by 3.3 minutes (p=0.8). A positive/stable total sleeping time emphasis the finding in sleep efficiency, because sleep efficiency is total sleeping time divided by total time in bed. After 13-14 days, the treatment difference of wake up after sleep onset, as well as of total time in bed were significant decreased by approx. 15 minutes (p=0.08), 50 minutes (p=0.048), respectively. (Table 2 and 3).

TABLE 2
Treatment difference of wake after sleep onset
in (min) estimated by a mixed model.

| days | Delta | Lo | Up | p-value |
|---|---|---|---|---|
| 1-3 | 0.4 | −16.1 | 16.9 | 0.96 |
| 4-6 | 0.1 | −16.4 | 16.6 | 0.99 |
| 13-14 | −14.8 | −31.3 | 1.7 | 0.078 |

TABLE 3
Treatment difference of total time in bed
in (min) estimated by a mixed model.

| days | Delta | Lo | Up | p-value |
|---|---|---|---|---|
| 1-3 | 15.4 | −34.6 | 65.3 | 0.54 |
| 4-6 | −40.6 | −90.6 | 9.3 | 0.11 |
| 13-14 | −50.5 | −100.5 | −0.6 | 0.048 |

Overall, the sleep actigraphy findings for sleep quality suggest an improvement in sleep efficiency by faster falling asleep and less wake up time during the night.

Also, results showed that the treatment statistically decreased the Karolinska Sleepiness Scale (KSS) measuring an at day sleepiness (Delta=−0.46; p=0.002) indicating an effect of product intake on the secondary effect of improved sleep quality.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of improving sleep and/or subsequent behavioural outcomes of an individual in need thereof by a device having one or more processors, the method comprising:
receiving an input from the individual, wherein the input comprises at least one of sleep quality, gender, age, body mass index, physical activity level, glycemic status, food preference, or food sensitivity,
evaluating, by the device, a need of the individual, wherein the need is selected from the group consisting of improving sleep quality, improving falling asleep, improving sleep maintenance, maintaining sleep quality, and combinations thereof, and
generating, by the device via an application interface and based on the input from the individual, a nutritional rule and the need of the individual, a recommendation for the individual, wherein the recommendation comprises at least one of a dietary plan comprising at least one of a recipe, a food item, a food product, or a diet tip, a dietary restriction on at least one of food sensitivity, sleep complain, or sleep comorbidity, a diet optimization of at least one of caloric intake, nutrient, meal glycemic load, or time of consumption, or sleep enhancing recommendation.

2. The method of claim 1 further comprising assessing the individual's sleep quality.

3. The method of claim 1, wherein the input comprises sleep quality,
the method further comprising
adopting the recommendation by the individual; and
reassessing, by the device, the individual's sleep quality.

4. The method of claim 1, wherein the dietary plan is adapted to the individual's preference selected from the group consisting of vegan, vegetarian, lactose-free, gluten-free and combination thereof.

5. The method of claim 1, wherein the recommendation comprises a multi-day evening meals plan.

6. The method of claim 1, wherein the nutritional rule comprises at least one of
(i) an amount and/or profile and/or a food source of a nutrient effective for promoting sleep, and the nutrient is selected from the group consisting of carbohydrate, tryptophan, magnesium, zinc, tryptophan, B-vitamins, melatonin, glycine, protein, PUFAs, omega 3, and combinations thereof, or
(ii) a glycemic load of a dietary intake.

7. The method of claim 1, wherein the recommendation comprises a sleep-enhancing evening meal to be consumed with a nutritional composition.

8-14. (canceled)

15. The method of claim 7, wherein the nutritional composition comprises at least one of a glucosidase inhibitor or an amylase inhibitor, the glucosidase inhibitor is selected from the group consisting of 1-deoxynojirimycin (DNJ), mulberry leaf extract, mulberry fruit extract, phloridzin, arginine-proline (AP) dipeptide and combinations thereof, and the amylase inhibitor is selected from the group consisting of white kidney bean extract, wheat albumin and combinations thereof.

16. A method of improving sleep and/or subsequent behavioural outcomes of an individual in need thereof, the method comprising:
providing a dietary recommendation by evaluating a parameter of the individual selected from the group consisting of falling asleep time, sleep duration, sleep onset latency, wakeafter sleep onset, sleep efficiency, gender, age, body mass index, physical activity level, glycemic status, food preference, food sensitivity, caloric intake, nutrients, meal glycemic load, time of consumption and combinations thereof,
providing a nutritional composition based on the dietary recommendation, and administering the nutritional composition to the individual,
wherein the nutritional composition is selected from the group consisting of a first composition comprising alpha-casozepine, a second composition comprising tryptophan, a third composition comprising mulberry leaf extract, and combinations thereof.

17. The method of claim 16, wherein the dietary recommendation comprises a recommendation for a meal comprising a nutrient selected from the group consisting of magnesium, zinc, tryptophan, B-vitamins, melatonin, glycine, protein, healthy fats, omega 3, fiber and combinations thereof.

18. The method of claim 16, wherein the nutritional composition is administered to the individual at a predetermined time before consumption of a meal.

19. (canceled)

20. The method of claim 16, wherein the nutritional composition is administered to the individual concurrently with consumption of a meal.

21. (canceled)

22. The method of claim 16, wherein the individual is selected from the group consisting of a child, an adolescent, an adult, and an elderly person.

23. The method of claim 16, wherein the dietary recommendation is adapted to an individual preference selected from the group consisting of vegan, vegetarian, lactose-free, gluten-free and combination thereof.

24. The method of claim 16, wherein the dietary recommendation includes a single day meal plan.

25. The method of claim 16, wherein the dietary recommendation includes a multi-day meal plan.

26. The method of claim 16, wherein the nutritional composition comprises the first composition and is administered along with a meal comprising at least one of (i) tryptophan from about 120 mg up to about 5 g, (ii) a sleep initiation beneficial micronutrient selected from the group consisting of magnesium, zinc, B-vitamins and combinations thereof, or (iii) a sleep functional ingredient is selected from the group consisting of melatonin, gamma aminobutyric acid (GABA), herbal extracts and combinations thereof.

27. The method of claim 16, wherein the nutritional composition comprises at least one of the second composition or the third composition and is administered along with an evening meal.

28. (canceled)

29. The method of claim 16, wherein the nutritional composition comprises at least one of a glucosidase inhibitor or an amylase inhibitor, the glucosidase inhibitor is selected from the group consisting of 1-deoxynojirimycin (DNJ), mulberry leaf extract, mulberry fruit extract, phloridzin, arginine-proline (AP) dipeptide and combinations thereof, and the amylase inhibitor is selected from the group consisting of white kidney bean extract, wheat albumin and combinations thereof.

30. A non-transitory computer-readable medium storing a program for improving sleep and/or subsequent behavioural outcomes of an individual in need thereof, that causes a device to:
provide a dietary recommendation by evaluating a parameter of the individual selected from the group consisting of falling asleep time, sleep duration, sleep onset latency, wakeafter sleep onset, sleep efficiency, gender, age, body mass index, physical activity level, glycemic status, food preference, food sensitivity, caloric intake, nutrients, meal glycemic load, time of consumption and combinations thereof, and
order a nutritional composition based on the dietary recommendation,
wherein the nutritional composition is selected from the group consisting of a first composition comprising alpha-casozepine, a second composition comprising tryptophan, a third composition comprising mulberry leaf extract and combination thereof.

31-39. (canceled)
